(19) United States
(12) Patent Application Publication
(10) Pub. No.: US 2023/0094597 A1
(43) Pub. Date: Mar. 30, 2023

(54) COMPOSITIONS AND METHODS FOR TREATING DISORDERS CHARACTERIZED WITH EXCESSIVE OSTEOCLAST ACTIVITY

(71) Applicant: THE JOHNS HOPKINS UNIVERSITY, Baltimore, MD (US)

(72) Inventor: Xu Cao, Baltimore, MD (US)

(21) Appl. No.: 17/934,937

(22) Filed: Sep. 23, 2022

Related U.S. Application Data
(60) Provisional application No. 63/248,135, filed on Sep. 24, 2021.

(51) Int. Cl. A61K 38/47; C07K 16/28; A61P 19/08

(52) U.S. Cl. CPC A61K 38/47; C07K 16/2875; A61P 19/08; C07K 2317/21; A61K 39/00

(57) ABSTRACT

Provided herein are compositions and methods directed to treating, delaying progression of, or reducing cancer growth or metastasis, and the severity of disorders characterized with increased osteoclast activity through hindering and/or inhibiting osteoclastogenesis and/or osteoclast activity. In particular, the present invention provides compositions and methods directed to hindering and/or inhibiting Siglec15 and sialylated TLR (e.g., TLR2, TLR4) interaction by specific sialyltransferases.

### CROSS-REFERENCE TO RELATED APPLICATION

This application claims priority to U.S. Provisional Patent Application No. 63/248,135 filed Sep. 24, 2021, the entire contents of which are hereby incorporated by reference in its entirety.

### FEDERALLY SPONSORED RESEARCH OR DEVELOPMENT

This invention was made with government support under AR071432 awarded by the National Institutes of Health. The government has certain rights in this invention.

### SEQUENCE LISTING

The text of the computer readable sequence listing filed herewith, titled “JHU-38723-203”, created Sep. 23, 2022, having a file size of 15,000 bytes, is hereby incorporated by reference in its entirety.

### FIELD OF THE INVENTION

Provided herein are compositions and methods directed to treating, delaying progression of, or reducing cancer growth or metastasis, and the severity of disorders characterized with increased osteoclast activity through hindering and/or inhibiting osteoclastogenesis and/or osteoclast activity. In particular, the present invention provides compositions and methods directed to hindering and/or inhibiting Siglec15 and sialylated TLR (e.g., TLR2, TLR4) interaction by specific sialyltransferases.

### INTRODUCTION

Blocking activation of T cells and/or macrophages promotes tumor progression and metastasis. TLRs, such as TLR2 and TLR4 are expressed in both T cells and macrophages for their activation. Sialilation of the TLRs blocks activation of T cells and macrophages by their agonists by binding Siglect15. Tumors also express TLRs and Siglect15 to block activation of T cells and macrophages for their progression and metastasis.

Disorders characterized with excessive osteoclast activity, such as osteoporosis, Paget's disease, rheumatoid arthritis, osteoclastoma, and periprosthetic osteolysis, are currently the most common reasons for bone inflammation, pain and fractures, resulting in low quality of life. However, the curative effects of current therapeutic drugs for these osteoclast-related diseases are limited, and long-term treatment is needed. Further, in severe cases, surgical treatments are necessary, which may cause unaffordable expenses and subsequent influences such as neuralgia, mental stress, and even development of cancer.

Thus, safer inhibitors and potential drugs with enhanced curative effects and quick relief are needed to treat patients with osteoclast diseases.

Thus, there is a critical need for improved treatments for disorders characterized with excessive osteoclast activity.

The present invention addresses this need.

### SUMMARY OF THE INVENTION

Siglec15 suppresses T cell activation and antibody against Siglec15 has been used for cancer immunotherapy. However, Siglec15 is expressed in different tumors and macrophages, and its receptors on T cells are not known. Experiments conducted during the course of identifying embodiments for the present invention identified siglec15 receptors. It was shown that siglec15 binds to TLR2 and TLR4 only when they are specifically sialylated by sialyltransfereases such as ST3Gal1. Binding of TLRs to their ligands or agonists activates T cells as immunotherapy for cancer treatments. The binding of siglec15 or sialylation of TLRs alone could block activation of T cells. Therefore, the discovery of sialylation of TLRs and binding of siglec15 reveals an opportunity to activate T cells for cancer immunotherapy.

In tumor microenvironment, macrophages are changed to tumor-associated macrophages (TAM) to promote tumor growth and metastasis. However, the molecular mechanism of TAM formation is still under extensive investigation. Experiments conducted during the course of identifying embodiments for the present invention identified a cell recognition pattern distinguishing self from non-self as a prerequisite for further cell fusion. It was shown that osteoclast precursor cell recognition is mediated by Siglec15-TLRs such as TLR2 and TLR4 interaction. The expression of Siglec15 in macrophages is activated by M-CSF. Siglec15 recognizes sialylated TLR2, in which the sialic acid was transferred by ST3Gal1 stimulated by RANKL. Both Siglec15 specific deletion in TRAP-positive mononuclear cells or intrafemoral injection of sialidase reduced formation of osteoclasts and increased bone volume. Thus, these results uncovered that sialylation of TLR2 binding to Siglec15 between tumor cells and macrophages induces polarization of macrophages changing to TAMs, and inhibition of the sialylation or interaction will reduce TAMs and tumor growth.

Macrophages are involved in the detection, and phagocytosis of harmful foreign organisms including tumor cells. They can then present the antigens to T cells and initiate inflammation by releasing cytokines that activate other cells. Osteoclast multinucleation marked by cell fusion is the key to regulate osteoclast function and related bone disorders. However, the control mechanism in initiation of phagocytosis of tumor cells or cell fusion of TRAP-positive mononuclear cells for osteoclast formation remains unknown. sialylation of TLR2 binding to Siglec15 between macrophages as cell-fusion signal controls of cell-cell fusion for the balance between preosteoclasts and osteoclasts for bone homeostasis. Importantly, as tumor cells express Siglec15, TLRs such as TLR2 and TLR4 in the macrophages binding to Siglec15 of tumor cells initiates downstream signaling of both Siglec15 and TLRs as a mechanism of phagocytosis for tumor cells. However, in the tumor microenvironment, the interaction could lead to TAMs. Blocking the interaction TLR2 with Siglec15 between macrophages and tumor cells would reduce TAMs.

The present invention contemplates that reducing or inhibiting Siglec15 and sialylated TLR (e.g., TLR2, TLR4) interaction within specific cells (e.g., tumor cells, mononuclear cells) satisfies an unmet need for the treatment of disorders characterized with increased osteoclast activity and/or increased Siglec15 and sialylated TLR (e.g., TLR2, TLR4) interaction.

Additional experiments conducted during the course of developing embodiments for the present invention determined that, in rheumatoid arthritis, endogenous ST3Gal4, but not ST3Gal1 is significantly increased, whereas in ankylosing spondylitis, both ST3Gal4 and ST3Gal1 expression levels are increased. As such, it was concluded that inhibition of ST3Gal1 could be an effective therapeutic target for cancer and osteoporosis, inhibition of ST3Gal4 for rheumatoid arthritis, and inhibition of both ST3Gal4 and ST3Gal1 for ankylosing spondylitis.

Accordingly, in certain embodiments, the present invention provides compositions and methods directed to treating, delaying progression of, or reducing tumor progression, metastasis, and the severity of disorders characterized with increased osteoclast activity through hindering and/or inhibiting osteoclastogenesis and/or osteoclast activity. In particular, the present invention provides compositions and methods directed to hindering and/or inhibiting Siglec15 and sialylated TLR (e.g., TLR2, TLR4) interaction.

In certain embodiments, the present invention provides methods for treating, delaying progression of, or reducing tumor progression, metastasis, and the severity of disorders characterized with increased osteoclast activity and/or increased Siglec15 and sialylated TLR (e.g., TLR2, TLR4) interaction. Such methods are not limited to particular type or manner of treating, delaying progression of, or reducing the severity of disorders characterized with increased osteoclast activity and/or increased Siglec15 and sialylated TLR (e.g., TLR2, TLR4) interaction.

In some embodiments, the present invention provides methods for treating, delaying progression of, or reducing tumor progression, metastasis, and the severity of disorders characterized with increased osteoclast activity and/or increased Siglec15 and sialylated TLR (e.g., TLR2, TLR4) interaction comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, such administration results in one or more of the following: inhibition of Siglec15 activity and/or expression; inhibition of osteoclast precursor activation of Siglec15 expression; inhibition of M-CSF activity and/or expression thereby preventing osteoclast precursor activation of Siglec15 expression; inhibition of sialyation of TLR (e.g., TLR2, TLR4) thereby inhibiting interaction between Siglec15 and TLR (e.g., TLR2, TLR4); inhibition of RANKL stimulation of a sialytransferase (e.g., ST3Gal1, ST3Gal4) thereby inhibiting transfer of sialic acid to TLR (e.g., TLR2, TLR4) from such a sialytransferase (e.g., ST3Gal1, ST3Gal4); and inhibition of transfer of sialic acid to TLR (e.g., TLR2, TLR4) from a sialytransferase (e.g., ST3Gal1, ST3Gal4).

Such methods are not limited to particular types or kinds of disorders characterized with increased osteoclast activity and/or increased Siglec15 and sialylated TLR (e.g., TLR2, TLR4) interaction. In some embodiments, the disorders characterized with increased osteoclast activity and/or increased Siglec15 and sialylated TLR (e.g., TLR2, TLR4) interaction include, but are not limited to, tumor progression, metastasis, osteoporosis, rheumatoid arthritis, bone destruction accompanying rheumatoid arthritis, hypercalcemia, hypocalcemia, cancerous hypercalcemia, bone destruction accompanying multiple myeloma or cancer metastasis to bone, giant cell tumor, tooth loss due to periodontitis, osteolysis around a prosthetic joint, osteomyelitis, Paget's disease, ankylosing spondylitis, renal osteodystrophy, osteogenesis imperfecta, childhood osteoporosis, osteomalacia, bone necrosis, metastatic bone diseases, myeloma, fibrous dysplasia, aplastic bone diseases, metabolic bone diseases, and bone loss with age.

Such embodiments are not limited to a particular type of agent capable of hindering and/or inhibiting Siglec15 and sialylated TLR (e.g., TLR2, TLR4) interaction (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.). In some embodiments, the agent is capable of one or more of the following effects: inhibition of Siglec15 activity and/or expression; inhibition of osteoclast precursor activation of Siglec15 expression; inhibition of M-CSF activity and/or expression thereby preventing osteoclast precursor activation of Siglec15 expression; inhibition of sialyation of TLR (e.g., TLR2, TLR4) thereby inhibiting interaction between Siglec15 and TLR (e.g., TLR2, TLR4); inhibition of RANKL stimulation of a sialytransferase (e.g., ST3Gal1, ST3Gal4) thereby inhibiting transfer of sialic acid to TLR (e.g., TLR2, TLR4) from such a sialytransferase (e.g., ST3Gal1, ST3Gal4); and inhibition of transfer of sialic acid to TLR (e.g., TLR2, TLR4) from a sialytransferase (e.g., ST3Gal1, ST3Gal4). In some embodiments, the agent capable of hindering and/or inhibiting Siglec15 and sialylated TLR (e.g., TLR2, TLR4) interaction is a sialytransferase inhibitor (e.g., soyasaponin I, AL-10, lithocholic acid (CAS: 434-13-9), lithocholylglycine (CAS: 474-74-8) lithocholyltaurine (CAS: 516-90-5), GK80030 (agilent.com/store/en_US/Prod-GK80030/GK80030), and GK80021 (agilent.com/store/en_US/Prod-GK80021/GK80021).

As noted, additional experiments conducted during the course of developing embodiments for the present invention determined that, in rheumatoid arthritis, endogenous ST3Gal4, but not ST3Gal1 is significantly increased, whereas in ankylosing spondylitis, both ST3Gal4 and ST3Gal1 expression levels are increased. As such, it was concluded that inhibition of ST3Gal1 could be an effective therapeutic target for cancer and osteoporosis, inhibition of ST3Gal4 for rheumatoid arthritis, and inhibition of both ST3Gal4 and ST3Gal1 for ankylosing spondylitis.

As such, in certain embodiments, the present invention provides methods for treating, delaying progression of, or reducing tumor progression, metastasis, and the severity of rheumatoid arthritis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal4 (e.g., endogenous ST3Gal4) expression and/or activity.

In certain embodiments, the present invention provides methods for treating, delaying progression of, or reducing tumor progression, metastasis, and the severity of osteoporosis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal1 (e.g., endogenous ST3Gal1) expression and/or activity.

In certain embodiments, the present invention provides methods for treating, delaying progression of, or reducing the severity of cancer (e.g., cancer characterized with increased Siglec15 and sialylated TLR (e.g., TLR2, TLR4) interaction) comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal1 (e.g., endogenous ST3Gal1) expression and/or activity.

In certain embodiments, the present invention provides methods for treating, delaying progression of, or reducing the severity of ankylosing spondylitis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting both ST3Gal1 (e.g., endogenous ST3Gal1) and ST3Gal4 (e.g., endogenous ST3Gal4) expression and/or activity. In some embodiments, the subject is given an agent is capable of inhibiting ST3Gal1 (e.g., endogenous ST3Gal1) and an agent capable of inhibiting ST3Gal4 (e.g., endogenous ST3Gal4) expression and/or activity.

In any of the described embodiments, the agent is formulated to be administered in any desirable manner (e.g., locally, orally, systemically, intravenously, intraarterially, subcutaneously, or intrathecally).

In certain embodiments of the invention, combination treatment with the agent of hindering and/or inhibiting Siglec15 and sialylated TLR (e.g., TLR2, TLR4) interaction and a course of a drug known for treating disorders characterized with increased osteoclast activity and/or increased Siglec15 and sialylated TLR (e.g., TLR2, TLR4) interaction (e.g., a drug known for treating osteoporosis and related disorders; a drug known for treating rheumatoid arthritis; a drug known for treating cancer; a drug known for treating ankylosing spondylitis).

The invention also provides pharmaceutical compositions comprising the agent capable of hindering and/or inhibiting Siglec15 and sialylated TLR (e.g., TLR2, TLR4) interaction in a pharmaceutically acceptable carrier.

Such methods are not limited to a specific meaning for a therapeutically effective amount of an agent capable of hindering and/or inhibiting Siglec15 and sialylated TLR (e.g., TLR2, TLR4) interaction. In some embodiments, a therapeutically effective amount of an agent capable of hindering and/or inhibiting Siglec15 and sialylated TLR (e.g., TLR2, TLR4) interaction is any dosage amount and duration that accomplishes is effective in treating, delaying progression of, or reducing the severity of disorders characterized with increased osteoclast activity and/or increased Siglec15 and sialylated TLR (e.g., TLR2, TLR4) interaction.

Such methods are not limited to specific osteoclast cells (e.g., precursor osteoclast cells and mature osteoclast cells). In some embodiments, the osteoclast cells are any type or kind of osteoclast cell expressing and/or capable of expressing Siglec15. In some embodiments, the osteoclast cells are any type or kind of osteoclast cell having Siglec15 activity.

In some embodiments, the subject is a mammalian subject (e.g., mouse, horse, human, cat, dog, gorilla, chimpanzee, etc.). In some embodiments, the subject is a human patient suffering from or at risk of suffering from a disorder characterized with increased osteoclast activity and/or increased Siglec15 and sialylated TLR (e.g., TLR2, TLR4) interaction.

In certain embodiments, the present invention provides methods of inhibiting and/or reducing Siglec15 activity and/or expression comprising exposing cells (e.g., osteoclast cells) (e.g., tumor cells) (e.g., in vivo, in vitro, in situ, ex vivo) characterized with increased Siglec15 activity and/or expression (compared to an established normal activity and/or expression level) a therapeutically effective amount of an agent configured to inhibit and/or diminish Siglec15 activity and/or expression within osteoclast cells. Such embodiments are not limited to a particular type of agent capable of inhibiting Siglec15 activity and/or expression (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing osteoclast precursor activation of Siglec15 expression through inhibiting M-CSF activity and/or expression comprising exposing cells (e.g., in vivo, in vitro, in situ, ex vivo) (e.g., osteoclast cells) (e.g., tumor cells) characterized with increased M-CSF activity and/or expression (compared to an established normal activity and/or expression level) a therapeutically effective amount of an agent configured to inhibit and/or diminish M-CSF activity and/or expression within osteoclast cells. Such embodiments are not limited to a particular type of agent capable of inhibiting M-CSF activity and/or expression (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing sialyation of TLR (e.g., TLR2, TLR4) comprising exposing cells (e.g., in vivo, in vitro, in situ, ex vivo) (e.g., osteoclast cells) (e.g., tumor cells) characterized with increased TLR (e.g., TLR2, TLR4) sialylation (compared to an established normal activity and/or expression level) a therapeutically effective amount of an agent configured to inhibit and/or diminish TLR (e.g., TLR2, TLR4) sialylation within osteoclast cells. Such embodiments are not limited to a particular type of agent capable of inhibiting TLR (e.g., TLR2, TLR4) sialylation (e.g., antibody, mimetic, siRNA molecule, small molecule, etc.). In some embodiments, the agent is a sialidase capable of inhibiting sialyation of TLR (e.g., TLR2, TLR4), and thereby inhibiting interaction between Siglec15 and TLR (e.g., TLR2, TLR4). Such embodiments are not limited to a particular type of sialidase (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing RANKL stimulation of a sialytransferase (e.g., ST3Gal1, ST3Gal4) comprising exposing cells (e.g., in vivo, in vitro, in situ, ex vivo) (e.g., osteoclast cells) (e.g., tumor cells) characterized with increased RANKL activity and/or expression (compared to an established normal activity and/or expression level) a therapeutically effective amount of an agent configured to inhibit and/or diminish RANKL stimulation of a sialytransferase (e.g., ST3Gal1, ST3Gal4) within osteoclast cells. Such embodiments are not limited to a particular type or kind of agent capable of inhibiting RANKL stimulation of a sialytransferase (e.g., ST3Gal1, ST3Gal4) (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.). In some embodiments, such an agent is selected from denosumab.

In certain embodiments, the present invention provides methods of inhibiting and/or reducing transfer of sialic acid to TLR (e.g., TLR2, TLR4) from a sialytransferase (e.g., ST3Gal1, ST3Gal4) comprising exposing cells (e.g., in vivo, in vitro, in situ, ex vivo) (e.g., osteoclast cells) (e.g., tumor cells) characterized with increased TLR sialyation (compared to an established normal activity and/or expression level) a therapeutically effective amount of an agent configured to inhibit and/or diminish transfer of sialic acid to TLR (e.g., TLR2, TLR4) from a sialytransferase (e.g., ST3Gal1, STE3Gal4) within osteoclast cells. Such embodiments are not limited to a particular type or kind of agent capable of inhibiting transfer of sialic acid to TLR (e.g., TLR2, TLR4) from a sialytransferase (e.g., ST3Gal1, STE3Gal4) (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.). For cancer treatment, such therapeutic methods can be used in combination with different cancer immunotherapies such as PD1/PDL1, Siglec15 therapies.

The invention also provides kits comprising one or more capable of hindering and/or inhibiting Siglec15 and sialylated TLR (e.g., TLR2, TLR4) interaction and instructions for administering the agent to an animal. The kits may optionally contain one or more other therapeutic agents.

### BRIEF DESCRIPTION OF DRAWINGS

FIG. 1A-J. TRAP⁺ pOC fusion was reduced in Siglec-15 deficiency mice with increase of bone volume. (A) Illustration of preparation for TRAP⁺ pOC and mOC differentiation. (B) Hierarchical clustering heat map showing expression of mouse Siglec family in BMM, pOC and mOC were detected and analysis by RNA-seq. (C) Western blot analysis of Siglec15 in BMM, pOC and mOC. (D) Representative μCT images of femurs from Siglec15^{ﬂ/ﬂ} and Siglec15^{ΔLysM} mice, 3D trabecular reconstruction (upper) and 2D cross-sectional view (lower). (E) Quantitative μCT analysis of trabecular number (Tb.N), trabecular thickness (Tb.Th), trabecular separation (Tb.Sp) and cortical thickness (Ct.Th), n=5. (F) TRAP staining of distal Siglec15^{ﬂ/ﬂ} and Siglec15^{ΔLysM} femur sections and quantification of multinuclear osteoclast number, bar represents 200 μm. TRAP staining with quantification of osteoclast number per well (G) and relative TRAP activity (H) of isolated BMMs from Siglec15^{ﬂ/ﬂ} and Siglec15^{ΔLysM} mice. (I) Immunostaining of phalloidin and Siglec15 of Siglec15^{ﬂ/ﬂ} (WT) and Siglec15^{ΔLysM} osteoclast with quantification of average nuclei number. (J) Bone resorption assay of Siglec15 of Siglec15^{ﬂ/ﬂ} (WT) and Siglec15^{ΔLysM} osteoclast quantified by pit area formation, n=3. Bar represents 100 μm. Data represents Mean±SD, statistically significant differences are indicated as*(p<0.05), ** (p<0.01), *** (p<0.001).

FIG. 2. TRAP stain of distal 11 wk-old Siglec15^{ﬂ/ﬂ} (WT) and Siglec15^{ΔLysM} mouse femur sections and quantification of multinuclear osteoclast number, n=3. Bar represents 200 μm. Data represents Mean±SD, statistically significant differences are indicated as ** (p<0.01).

FIG. 3A-B. Immunofluoresent stain of Siglec15 and TRAP of distal 4 wk-old (A) and 11 wk-old (B) Siglec15^{ﬂ/ﬂ} (WT) and Siglec15^{ΔLysM} mouse femur sections and quantification of osteoclast size, n=3. Bar represents 200 μm. Data represents Mean±SD, statistically significant differences are indicated as *** (p<0.001).

FIG. 4A-G. M-CSF induced transcription of Siglec15 via MEK-ERK-MYC signaling. (A) BMMs incubated with biotinylated Siglec15 monoclonal antibody were manually separated to Siglec15⁺BMMs and Siglec15⁻ BMMs using anti-biotin microbeads and magnetic separators. (B) Flow cytometry analysis of WT and separated Siglec15⁻ BMMs on day 0 and day 3 after treatment of M-CSF. (C) Quantification of Siglec15⁺cell proportion. (D) TRAP stain of WT, Siglec15⁻ and Siglec15⁺BMMs treated with M-CSF+ RANKL or RANKL alone. (E) Site-directed mutagenesis of the MYC binding sites at Siglec15 core enhancer. (F) Quantification of osteoclast number per well. (G) Quantification of mean Siglec15 fluorescent intensity was shown on the right. n=3. Data represents Mean±SD, statistically significant differences are indicated as ** (p<0.01), *** (p<0.001).

FIG. 5A-B. (A) TRAP staining of Siglec15⁺ and Siglec15- BMMs induced by RANKL and M-CSF. Bar represents 50 μm. (B) Quantification of osteoclast number per well, n=3. Data represents Mean±SD.

FIG. 6. TRAP stain of Siglec5^{ΔLysM} BMMs introduced with Siglec15⁺ BMMs of different proportion (0, 1%, 5%, 10%). Quantification of osteoclast number per well is shown below. n=3. Data represents Mean±SD.

FIG. 7A-B. (A) TRAP stain of Siglec15^{ΔLysM} BMMs in control wells or transwells co-incubated with Siglec15⁺ BMMs. Quantification of osteoclast number per well is shown below. Bar represents 50 μm. n=3. Data represents Mean±SD. (B) TRAP stain of Siglec15-BMMs in control wells or transwells co-incubated with Siglec15⁺ BMMs. Quantification of osteoclast number per well is shown below. Bar represents 50 μm. n=3. Data represents Mean±SD.

FIG. 8A-B. (A) Immunostaining of Siglec15 and phalloidin in WT, Siglec15⁻, Siglec15⁺, and Siglec15^{ΔLysM} BMMs during osteoclastogenesis. Bar represents 50 μm. (B) Quantification of average nuclei number and mean siglec15 fluorescent intensity. Data represents Mean±SD, statistically significant differences are indicated as *** (p<0.001).

FIG. 9A-B. (A) TRAP stain of WT, Siglec15⁻, Siglec15⁺, and Siglec15^{ΔLysM} BMMs induced with RANKL+M-CSF or RANKL+GM-CSF. (B) Quantification of osteoclast number per well on the right. n=3. Data represents Mean±SD, statistically significant differences are indicated as *** (p<0.001).

FIG. 10. Immunostaining of phalloidin and Siglec15 in Siglec15, Siglec15⁺, and Siglec15^{ΔLysM} BMMs treated with GM-CSF. Quantification of mean Siglec15 fluorescence is shown on the right. Bar represents 50 μm. n=3. Data represents Mean±SD, statistically significant differences are indicated as *** (p<0.001).

FIG. 11A-F. M-CSF induced transcription of Siglec15 via MEK-ERK-MYC signaling. (A) Flow cytometry analysis of Siglec15^{ﬂ/ﬂ} (WT) and Siglec15^{ΔLysM} BMMs treated with M-CSF or vehicle for 3 days quantified by Siglec15⁺ cell proportion. (B) Immunostaining of phalloidin and Siglec15 in BMMs treated with M-CSF, M-CSF+U0126 or vehicle with quantification of Siglec15⁺ cells, bar represents 50 μm. (C) Three MYC binding sites at the Siglec15 promoter core enhancer region and chromatin immunoprecipitation (ChIP) assay validation of the MYC binding sites. (D) Western blot analysis of ERK, MYC phosphorylation, and Siglec15 in BMMs treated with M-CSF, M-CSF+U0126 or vehicle at 0, 24 h, 48 h and 72 h. (E) Site-directed mutagenesis of the MYC binding sites at Siglec15 core enhancer. (F) Quantification of normalized luciferase activity of luciferase gene reporter assay in BMMs transfected with c-Myc expression vector, n=5. Data represents Mean±SD, statistically significant differences are indicated as ** (p<0.01), *** (p<0.001).

FIG. 12. TRAP stain of WT BMMs treated with vehicle or U0126 during osteoclastogenesis. Bar represents 100 μm. Quantification of osteoclast number per well. n=3. Data represents Mean±SD, statistically significant differences are indicated as *** (p<0.001).

FIG. 13A-B. (A) Three MYC recognition sites at the Siglec15 core enhancer. (B) chromatin immunoprecipitation (ChIP) assay validation of the direct MYC binding induced by GM-CSF.

FIG. 14A-E. Sialylated TLR2 is the binding ligand for Siglec-15. (A) Identification of 318 potential Siglec15 binding proteins using proximity labeling method followed by LC-MS/MS. Number of membrane proteins and glycoproteins was shown. (B) Most highly enriched glyco-membrane protein evaluated by mascot score, peak area and unique peptides. (C) Hierarchical clustering heat map showed expression of mouse TLR family in BMM, pOC and mOC detected by RNA-seq. (D) Immunoblotting (IB) of Siglec15 in whole cell lysates or protein complex immunoprecipitated (IP) with TLR2, IB of TLR2 in whole cell lysates or protein complex IP with Siglec15 in indicated groups. (E) IB of TLR2 in whole cell lysates or protein complex IP with Siglec15 in Siglec15^{ΔLysM} or Siglec15^{ﬂ/ﬂ} (WT) BMMs testing sialidase effects.

FIG. 15A-E. WT BMMs were treated with vehicle or sialidase. Sialidase was treated at the beginning and TRAP stain was performed at 48 h (A) and 120 h (B). Sialidase was treated at 72 h and TRAP stain was performed at 120 h (C). Quantification of osteoclast number per well on the right. n=3. Sialidase was treated at the beginning and pit formation was detected at 120 h (D), sialidase was treated at 72 h and pit formation was detected at 120 h (E). n=3. Bar represents 100 μm. Data represents Mean±SD, statistically significant differences are indicated as *** (p<0.001).

FIG. 16A-G. RANKL induced expression of sialic acid transferase ST3Gal1 to sialylate TLR2. (A) Immunostaining of α(2,3) and α(2,6) sialic acid (SA) in WT BMMs with treatments as indicated. Quantification of fluorescent intensity. (B) Hierarchical clustering heat map showing expression of SA transferase family members in BMM, pOC and mOC were detected and analyzed by RNA-seq. (C) Immunostaining of α(2,3) with TLR2 or Siglec15 in Siglec15^{ﬂ/ﬂ} (WT) and Siglec15^{ΔLysM} BMMs. (D) Co-localization of α(2,3) with TLR2 or Siglec15. (E) FOS and CREB1 binding sites at the St3 gal1 promoter core enhancer. (F) ChIP assay validation of FOS and CREB binding activity. (G) Site-directed mutagenesis of FOS binding sites at St3 gal1 core enhancer. Quantification of normalized luciferase activity of luciferase gene reporter assay in BMMs transfected with c-Fos expression vector, n=5. Data represents Mean±SD, statistically significant differences are indicated as ** (p<0.01).

FIG. 17. ELISA detection of TNF-alpha, IL-6 and IL-1beta in WT BMMs treated as indicated. n=3. Data represents Mean±SD, statistically significant differences are indicated as *** (p<0.001).

FIG. 18. Immunohistochemistry (IHC) staining of ST3Gal1 in RANKL^{ﬂ/ﬂ} and RANKL^{ΔDmp} mice distal femurs. Bar represents 200 μm. Quantification of ST3Gal1 positive area is shown on the right. n=3. Data represents Mean±SD, statistically significant differences are indicated as*(p<0.05), *** (p<0.001).

FIG. 19A-B. (A) Immunostaining of α(2,3) and TLR2 of distal femur sections of RANKL^{ﬂ/ﬂ} and RANKL^{ΔDmp} mice and co-localization analysis of α(2,3) with TLR2. (B) Immunostaining of Siglec15 and TLR2 of distal femur sections of RANKL^{ﬂ/ﬂ} and RANKL^{ΔDmp} mice and co-localization analysis of Siglec15 with TLR2.

FIG. 20A-B. (A) TRAP staining of RAW264.7 cells transfected with St3gal1 siRNA or vehicle before RANKL stimulation for 72 h. (B) Quantification of osteoclast number per well, n=3. Data represents Mean±SD, statistically significant differences are indicated as *** (p<0.001).

FIG. 21A-F. Representative μCT cross-sectional femur images of Siglec15^{ﬂ/ﬂ} (WT) and Siglec15^{ΔLysM} mice intrafemorally injected with vehicle (VEH) or sialidase. The bar represents 4 mm. b Quantitative μCT analysis of trabecular number (Tb.N), trabecular thickness (Tb.Th), bone volume/tissue volume (BV/TV), and trabecular connectivity (Tb. Con); n=5. c TRAP staining of distal femur sections from Siglec15^{ﬂ/ﬂ} (WT) mice intrafemorally injected with vehicle or sialidase and quantification of the multinuclear osteoclast number, n=3. d Immunostaining of α2,3 SA and TLR2 in distal femur sections from Siglec15^{ﬂ/ﬂ} (WT) mice intrafemorally injected with vehicle or sialidase and colocalization analysis of α2,3 with TLR2. e Body weight and femur length of Siglec15^{ﬂ/ﬂ} (WT) and Siglec15^{ΔLysM} mice intrafemorally injected with vehicle or sialidase, n=5. f Schematic diagram showing the self/nonself-recognition mediated by siglec15 binding with sialylated TLR2 before further cell fusion. Data represent the mean±SD, and statistically significant differences are indicated as *P<0.05; **P<0.01; ***P<0.001

FIG. 22. TRAP staining of distal femur sections of Siglec15^{ΔLysM} mice intrafemorally injected with vehicle or sialidase and quantification of multinuclear osteoclast number, n=3.

FIG. 23. Relative expression of osteomorph marker genes Bpgm and Fbxo7 in Siglec15^{ﬂ/ﬂ} and Siglec15^{ΔLysM} mice with or without OPG:Fc treatments, n=5. Data represents Mean±SD, statistically significant differences are indicated as ** (p<0.01), *** (p<0.001).

FIG. 24 shows that RANKL induces St3gal1 expression is required for osteoclast formation.

FIG. 25A-D shows that bone sialylation and St3Gal1 levels are increased in estrogen deficient osteoporosis.

FIG. 26A-I shows that estrogen inhibits RANKL-activated St3gal1 transcription by repressing c-Fos Expression.

FIG. 27 shows that sialidase protects bone loss in OVX osteoporotic mice.

FIG. 28A-H shows that binding of Siglec15 and TLR2 activates both of their downstream signaling pathways. A. Trap+ mononuclear cells were marked with CellTracker Green, and BMMs with or without St3gal1 overexpression were marked with the cell label DiI. The cells were then cocultured for 24 or 72 h before observation using a fluorescence microscope. B. Quantification of the membrane merge rate in (a). C. Immunoblotting (IB) for DAP12 in whole-cell lysates or protein complexes immunoprecipitated (IP) with Siglec15 and IB for Siglec15 in whole-cell lysates or protein complexes IP with DAP12 in the indicated groups. D. IB for DAP12 and Syk in whole-cell lysates or protein complexes IP with p-Tyr in the indicated groups. e IB for MyD88 in protein complexes IP with TLR2 in the indicated groups. IB for TLR2, NFATc1, and Siglec15 in whole-cell lysates. F. Immunostaining with phalloidin (red) and anti-p65 (green) in the indicated groups to observe p65 nuclear translocation. Scale bar=20 μm. G. Quantification of the number and percentage of p65-positive nuclei. H. Western blot analysis of p-IκBα and p65 in the cytosol and p65 in the nucleus in the indicated groups. Data represent the mean±SD, and statistically significant differences are indicated as ***P<0.001

FIG. 29 shows that expression of both St3Gal1 and St3Gal4 are significantly elevated in human autoimmune diseases including rheumatoid arthritis and ankylosing spondylitis.

FIG. 30A-B shows sialylation is required for the binding between TLR2 and Siglec-15.

FIG. 31A-E shows sialidase blocks cell-cell fusion for osteoclast formation.

FIG. 32A-B shows sialyltransferase St3Gal1 is transcriptionally induced by RANKL.

FIG. 33 shows α(2, 3) sialic acid is co-localized with TLR2.

FIG. 34A-G shows Siglec15 expression is stimulated by M-CSF.

FIG. 35A-F shows M-CSF induced transcription of Siglec15 via MEK-ERK-MYC signaling.

FIG. 36 shows RANKL Induces St3gal1 expression through Fos binding at its promoter.

FIG. 37 shows St3 gal1 expression id abolished in RANKL^{ΔDmp} mice.

FIG. 38 shows siRNA St3gal1 blocked cell-cell fusion for osteoclast formation.

FIG. 39 shows sialidase inhibits RANKL effects on bone marrow macrophages.

FIG. 40A-B shows knockout of RANKL in DMP-1 osteocytes disrupted α(2, 3) sialic acid co-localization with TLR2 and Siglec15.

FIG. 41 shows removal of TLR2 α(2,3) sialic acid by sialidase increases bone mass in mice.

FIG. 42 shows removal of TLR2 α(2,3) sialic acid by sialidase increases bone mass in mice.

FIG. 43 shows a schematic diagram of Siglec15 binding to sialylated TLR2 for osteoclast fusion.

FIG. 44 shows sialyltransferase St3Gal4, not St3Gal1 is up-regulated in rheumatoid arthritis.

FIG. 45. Schematic diagram showing that Siglec15 is activated by M-CSF via ERK1/2-cMyc signaling and St3gal1 is activated by RANKL via c-Fos dependent pathway.

### DETAILED DESCRIPTION OF THE INVENTION

Macrophage/monocyte survival and proliferation are maintained by macrophage colony stimulating factor (M-CSF), and receptor activator of NF-κappaB ligand (RANKL) further promotes commitment to the osteoclast lineage as tartrate-resistant acid phosphatase-positive (Trap+) mononuclear cells, which are also known as preosteoclasts (Lacey, 1998; Yasuda, 1998). In an appropriate microenvironment, preosteoclasts subsequently undergo cell-cell fusion to form Trap+ multinuclear osteoclasts (Boyle, 2003; Koga, 2004). In particular, alterations in osteoclast differentiation or activity can result in almost every major skeletal disorder, such as osteoporosis, skeletal degeneration and pain, arthritis, and Paget disease. Loss-of-function mutations in or deletion of M-CSF causes severe osteopetrosis in patients with no osteoclast-lineage cells. Mice with a nullizygous M-CSF deletion (Csƒ1^{op}/Csƒ1^{op}) also develop osteopetrosis (Dai, 2002). In patients with a RANKL mutation, macrophage-lineage cells are not able to commit to differentiation into osteoclasts. Similarly, RANKL-deficient (Tnsƒ11^{-/-}) mice have problems in osteoclast differentiation with an osteopetrosis phenotype (Pettit, 2001). Both M-CSF and RANKL are needed for osteoclast formation, but neither of them can induce osteoclast formation alone. Together, M-CSF and RANKL effectively induce the expression of osteoclast lineage-specific genes and lead to the development of osteoclast maturation marked by cell fusion-mediated multinucleation (Lacey, 1998; Boyle, 2003). The requirement for cell fusion in osteoclast formation has been studied for decades, and numerous fusogenic molecules have been identified. However, the mechanism controlling the initiation of cell fusion during osteoclast formation remains unknown.

Toll-like receptors (TLRs) are critical in the innate immune response and function by recognizing pathogen-associated molecular patterns with myeloid differentiation primary response protein 88 (MyD88) as a main adaptor (Medzhitov, 2001). Trap* macrophages/mononuclear cells fuse with their own kind instead of a similar cell type and are also dependent on a self-recognition mechanism. Interestingly, TLRs have an inhibitory effect on osteoclast differentiation upon agonist stimulation (Takami, 2002). TLR signaling is regulated by various mechanisms, such as ectodomain modification of N-linked glycans orchestrating TLR signaling capacities. The removal of sialyl residues from TLR glycosylation sites after neuraminidase treatment is enhanced following agonist stimulation (Weber, 2002, Amith, 2010). This suggests that TLR function can potentially be blocked by sialylation to ensure the normal progress of osteoclast differentiation.

Sialylation is a process mediated by sialyltransferases (STs), which catalyze the transfer of a sialic acid (SA) moiety to various acceptors in different linkages. SAs compose a family of nine-carbon acidic monosaccharides on N— and O—linked glycans and are attached to galactose or N-acetylgalactosamine units via α2,3—or α2,6-linkages (Varki, 2008). Sialylated glycoconjugates are involved in various cellular events, such as cell adhesion, hematopoietic stem cell fate determination, and viral fusion (Crean et al., 2004; Keppler et al., 1999; Stamatos et al., 2004). SAs are specifically bound by SA-binding immunoglobulin-type lectins (Siglecs) that are primarily found on the surface of immune-related cells. Increases in SA have been widely implicated in different skeletal diseases (Vijay, 1982), and blockade of SA has been shown to be effective in suppressing tumor growth by enhancing CD8⁺ T-cell activation (Bull, 2018; Urban-Wojciuk, 2019).

Experiments conducted during the course of identifying embodiments for the present invention sought to characterize the molecular mechanism of cell recognition that initiates osteoclast fusion. It was found that TLR2 functioned as a Siglec15 receptor and that sialylation of TLR2 enabled osteoclast precursors to recognize themselves from nonself cells. Removal of sialic acid from TLR2 disabled cell recognition mediated by Siglec15 and inhibited consequential osteoclast fusion. This finding of osteoclast recognition signaling is helpful in understanding the pathogenesis of different skeletal disorders and bone loss during aging.

Additional experiments conducted during the course of developing embodiments for the present invention determined that, in rheumatoid arthritis, endogenous ST3Gal4, but not ST3Gal1 is significantly increased, whereas in ankylosing spondylitis, both ST3Gal4 and ST3Gal1 expression levels are increased. As such, it was concluded that inhibition of ST3Gal1 could be an effective therapeutic target for cancer and osteoporosis, inhibition of ST3Gal4 for rheumatoid arthritis, and inhibition of both ST3Gal4 and ST3Gal1 for ankylosing spondylitis.

Indeed, the molecular control of osteoclast formation is still not clearly elucidated. Such experiments described herein demonstrate that a process of cell recognition mediated by Siglec15-TLR2 binding is indispensable and occurs prior to cell fusion in RANKL-mediated osteoclastogenesis. Siglec15 has been shown to regulate osteoclastic bone resorption. However, the receptor for Siglec15 has not been identified, and the signaling mechanism involving Siglec15 in osteoclast function remained unclear. It was found that Siglec15 bound sialylated TLR2 as its receptor and that the binding of sialylated TLR2 to Siglec15 in macrophages committed to the osteoclast-lineage initiated cell fusion for osteoclast formation, in which sialic acid was transferred by the sialyltransferase ST3Gal1.

Interestingly, the expression of Siglec15 in macrophages was activated by M-CSF, whereas ST3Gal1 expression was induced by RANKL. Both Siglec15-specific deletion in macrophages and intrafemoral injection of sialidase abrogated cell recognition and reduced subsequent cell fusion for the formation of osteoclasts, resulting in increased bone formation in mice. Thus, these results reveal that cell recognition mediated by the binding of sialylated TLR2 to Siglec15 initiates cell fusion for osteoclast formation.

The present invention contemplates that reducing or inhibiting Siglec15 and sialylated TLR (e.g., TLR2, TLR4) interaction within specific cells (e.g., tumor cells, osteoclast cells) satisfies an unmet need for the treatment of disorders characterized with increased osteoclast activity and/or increased Siglec15 and sialylated TLR (e.g., TLR2, TLR4) interaction.

Accordingly, in certain embodiments, the present invention provides compositions and methods directed to treating, delaying progression of, or reducing cancer growth or metastasis, and the severity of disorders characterized with increased osteoclast activity through hindering and/or inhibiting osteoclastogenesis and/or osteoclast activity. In particular, the present invention provides compositions and methods directed to hindering and/or inhibiting Siglec15 and sialylated TLR (e.g., TLR2, TLR4) interaction by specific sialyltransferases.

In certain embodiments, the present invention provides methods for treating, delaying progression of, or reducing the severity of disorders characterized with increased osteoclast activity and/or increased Siglec15 and sialylated TLR (e.g., TLR2, TLR4) interaction. Such methods are not limited to particular type or manner of treating, delaying progression of, or reducing the severity of disorders characterized with increased osteoclast activity.

In some embodiments, the present invention provides methods for treating, delaying progression of, or reducing the severity of disorders characterized with increased osteoclast activity and/or increased Siglec15 and sialylated TLR (e.g., TLR2, TLR4) interaction comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, such administration results in one or more of the following: inhibition of Siglec15 activity and/or expression; inhibition of osteoclast precursor activation of Siglec15 expression; inhibition of M-CSF activity and/or expression thereby preventing osteoclast precursor activation of Siglec15 expression; inhibition of sialyation of TLR (e.g., TLR2, TLR4) thereby inhibiting interaction between Siglec15 and TLR (e.g., TLR2, TLR4); inhibition of RANKL stimulation of a sialytransferase (e.g., ST3Gal1, ST3Gal4) thereby inhibiting transfer of sialic acid to TLR (e.g., TLR2, TLR4) from such a sialytransferase (e.g., ST3Gal1, ST3Gal4); and inhibition of transfer of sialic acid to TLR (e.g., TLR2, TLR4) from a sialytransferase (e.g., ST3Gal1, ST3Gal4).

Such methods are not limited to particular types or kinds of disorders characterized with increased osteoclast activity and/or increased Siglec15 and sialylated TLR (e.g., TLR2, TLR4) interaction. In some embodiments, the disorders characterized with increased osteoclast activity and/or increased Siglec15 and sialylated TLR (e.g., TLR2, TLR4) interaction include, but are not limited to, osteoporosis, rheumatoid arthritis, bone destruction accompanying rheumatoid arthritis, hypercalcemia, hypocalcemia, cancerous hypercalcemia, bone destruction accompanying multiple myeloma or cancer metastasis to bone, giant cell tumor, tooth loss due to periodontitis, osteolysis around a prosthetic joint, osteomyelitis, Paget's disease, ankylosing spondylitis, renal osteodystrophy, osteogenesis imperfecta, childhood osteoporosis, osteomalacia, bone necrosis, metastatic bone diseases, myeloma, fibrous dysplasia, aplastic bone diseases, metabolic bone diseases, and bone loss with age.

Such embodiments are not limited to a particular type of agent capable of hindering and/or inhibiting Siglec15 and sialylated TLR (e.g., TLR2, TLR4) interaction (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.). In some embodiments, the agent is capable of one or more of the following effects: inhibition of Siglec15 activity and/or expression; inhibition of osteoclast precursor activation of Siglec15 expression; inhibition of M-CSF activity and/or expression thereby preventing osteoclast precursor activation of Siglec15 expression; inhibition of sialyation of TLR (e.g., TLR2, TLR4) thereby inhibiting interaction between Siglec15 and TLR (e.g., TLR2, TLR4); inhibition of RANKL stimulation of a sialytransferase (e.g., ST3Gal1, ST3Gal4) thereby inhibiting transfer of sialic acid to TLR (e.g., TLR2, TLR4) from such a sialytransferase (e.g., ST3Gal1, ST3Gal4); and inhibition of transfer of sialic acid to TLR (e.g., TLR2, TLR4) from a sialytransferase (e.g., ST3Gal1, ST3Gal4).

As noted, additional experiments conducted during the course of developing embodiments for the present invention determined that, in rheumatoid arthritis, endogenous ST3Gal4, but not ST3Gal1 is significantly increased, whereas in ankylosing spondylitis, both ST3Gal4 and ST3Gal1 expression levels are increased. As such, it was concluded that inhibition of ST3Gal1 could be an effective therapeutic target for cancer and osteoporosis, inhibition of ST3Gal4 for rheumatoid arthritis, and inhibition of both ST3Gal4 and ST3Gal1 for ankylosing spondylitis.

As such, in certain embodiments, the present invention provides methods for treating, delaying progression of, or reducing the severity of rheumatoid arthritis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal4 (e.g., endogenous ST3Gal4) expression and/or activity.

In certain embodiments, the present invention provides methods for treating, delaying progression of, or reducing the severity of osteoporosis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal1 (e.g., endogenous ST3Gal1) expression and/or activity.

In certain embodiments, the present invention provides methods for treating, delaying progression of, or reducing the severity of cancer (e.g., cancer characterized with increased Siglec15 and sialylated TLR (e.g., TLR2, TLR4) interaction) comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting ST3Gal1 (e.g., endogenous ST3Gal1) expression and/or activity.

In certain embodiments, the present invention provides methods for treating, delaying progression of, or reducing the severity of ankylosing spondylitis comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction. In some embodiments, the agent is capable of inhibiting both ST3Gal1 (e.g., endogenous ST3Gal1) and ST3Gal4 (e.g., endogenous ST3Gal4) expression and/or activity. In some embodiments, the subject is given an agent is capable of inhibiting ST3Gal1 (e.g., endogenous ST3Gal1) and an agent capable of inhibiting ST3Gal4 (e.g., endogenous ST3Gal4) expression and/or activity.

In any of the described embodiments, the agent is formulated to be administered in any desirable manner (e.g., locally, orally, systemically, intravenously, intraarterially, subcutaneously, or intrathecally).

In certain embodiments of the invention, combination treatment with the agent of hindering and/or inhibiting Siglec15 and sialylated TLR (e.g., TLR2, TLR4) interaction and a course of a drug known for treating disorders characterized with increased osteoclast activity and/or increased Siglec15 and sialylated TLR (e.g., TLR2, TLR4) interaction (e.g., a drug known for treating osteoporosis and related disorders; a drug known for treating rheumatoid arthritis; a drug known for treating cancer; a drug known for treating ankylosing spondylitis).

The invention also provides pharmaceutical compositions comprising the agent capable of hindering and/or inhibiting Siglec15 and sialylated TLR (e.g., TLR2, TLR4) interaction in a pharmaceutically acceptable carrier.

Such methods are not limited to a specific meaning for a therapeutically effective amount of an agent capable of hindering and/or inhibiting Siglec15 and sialylated TLR (e.g., TLR2, TLR4) interaction. In some embodiments, a therapeutically effective amount of an agent capable of hindering and/or inhibiting Siglec15 and sialylated TLR (e.g., TLR2, TLR4) interaction is any dosage amount and duration that accomplishes is effective in treating, delaying progression of, or reducing the severity of disorders characterized with increased osteoclast activity and/or increased Siglec15 and sialylated TLR (e.g., TLR2, TLR4) interaction.

Such methods are not limited to specific osteoclast cells (e.g., precursor osteoclast cells and mature osteoclast cells). In some embodiments, the osteoclast cells are any type or kind of osteoclast cell expressing and/or capable of expressing Siglec15. In some embodiments, the osteoclast cells are any type or kind of osteoclast cell having Siglec15 activity.

In certain embodiments, the present invention provides methods of inhibiting and/or reducing Siglec15 activity and/or expression comprising exposing cells (e.g., in vivo, in vitro, in situ, ex vivo) (e.g., osteoclast cells) (e.g., tumor cells) characterized with increased Siglec15 activity and/or expression (compared to an established normal activity and/or expression level) a therapeutically effective amount of an agent configured to inhibit and/or diminish Siglec15 activity and/or expression within osteoclast cells. Such embodiments are not limited to a particular type of agent capable of inhibiting Siglec15 activity and/or expression (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing osteoclast precursor activation of Siglec15 expression through inhibiting M-CSF activity and/or expression comprising exposing cells (e.g., in vivo, in vitro, in situ, ex vivo) (e.g., osteoclast cells) (e.g., tumor cells) characterized with increased M-CSF activity and/or expression (compared to an established normal activity and/or expression level) a therapeutically effective amount of an agent configured to inhibit and/or diminish M-CSF activity and/or expression within osteoclast cells. Such embodiments are not limited to a particular type of agent capable of inhibiting M-CSF activity and/or expression (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing sialyation of TLR (e.g., TLR2, TLR4) comprising exposing cells (e.g., in vivo, in vitro, in situ, ex vivo) (e.g., osteoclast cells) (e.g., tumor cells) characterized with increased TLR (e.g., TLR2, TLR4) sialylation (compared to an established normal activity and/or expression level) a therapeutically effective amount of an agent configured to inhibit and/or diminish TLR (e.g., TLR2, TLR4) sialylation within osteoclast cells. Such embodiments are not limited to a particular type of agent capable of inhibiting TLR (e.g., TLR2, TLR4) sialylation (e.g., antibody, mimetic, siRNA molecule, small molecule, etc.). In some embodiments, the agent is a sialidase capable of inhibiting sialyation of TLR (e.g., TLR2, TLR4), and thereby inhibiting interaction between Siglec15 and TLR (e.g., TLR2, TLR4). Such embodiments are not limited to a particular type of sialidase (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In certain embodiments, the present invention provides methods of inhibiting and/or reducing RANKL stimulation of a sialytransferase (e.g., ST3Gal1, ST3Gal4) comprising exposing cells (e.g., in vivo, in vitro, in situ, ex vivo) (e.g., osteoclast cells) (e.g., tumor cells) characterized with increased RANKL activity and/or expression (compared to an established normal activity and/or expression level) a therapeutically effective amount of an agent configured to inhibit and/or diminish RANKL stimulation of a sialytransferase (e.g., ST3Gal1, ST3Gal4) within osteoclast cells. Such embodiments are not limited to a particular type or kind of agent capable of inhibiting RANKL stimulation of a sialytransferase (e.g., ST3Gal1, ST3Gal4) (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.). In some embodiments, such an agent is selected from denosumab.

In certain embodiments, the present invention provides methods of inhibiting and/or reducing transfer of sialic acid to TLR (e.g., TLR2, TLR4) from a sialytransferase (e.g., ST3Gal1, ST3Gal4) comprising exposing cells (e.g., in vivo, in vitro, in situ, ex vivo) (e.g., osteoclast cells) (e.g., tumor cells) characterized with increased TLR sialyation (compared to an established normal activity and/or expression level) a therapeutically effective amount of an agent configured to inhibit and/or diminish transfer of sialic acid to TLR (e.g., TLR2, TLR4) from a sialytransferase (e.g., ST3Gal1, STE3Gal4) within osteoclast cells. Such embodiments are not limited to a particular type or kind of agent capable of inhibiting transfer of sialic acid to TLR (e.g., TLR2, TLR4) from a sialytransferase (e.g., ST3Gal1, STE3Gal4) (e.g., small molecule, a polypeptide or peptide fragment, an antibody or fragment thereof, a nucleic acid molecule (e.g., RNA, siRNA, microRNA, interference RNA, mRNA, replicon mRNA, RNA-analogues, and DNA), etc.).

In addition to administering the agent is administered as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The agents (e.g., agents capable of inhibiting Siglec15 and sialylated TLR (e.g., TLR2, TLR4) interaction) and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired.

Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention.

Various modifications and alterations of the compositions and methods described above can readily be achieved using expertise available in the art and are within the scope of the invention.

### EXPERIMENTAL

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention. Use of pronouns such as “I”, “we”, “our”, etc., is in reference to the inventors.

### Example I

This example demonstrates that Siglec15 deficiency in macrophage lineage cells leads to multinucleation failure and bone volume increase.

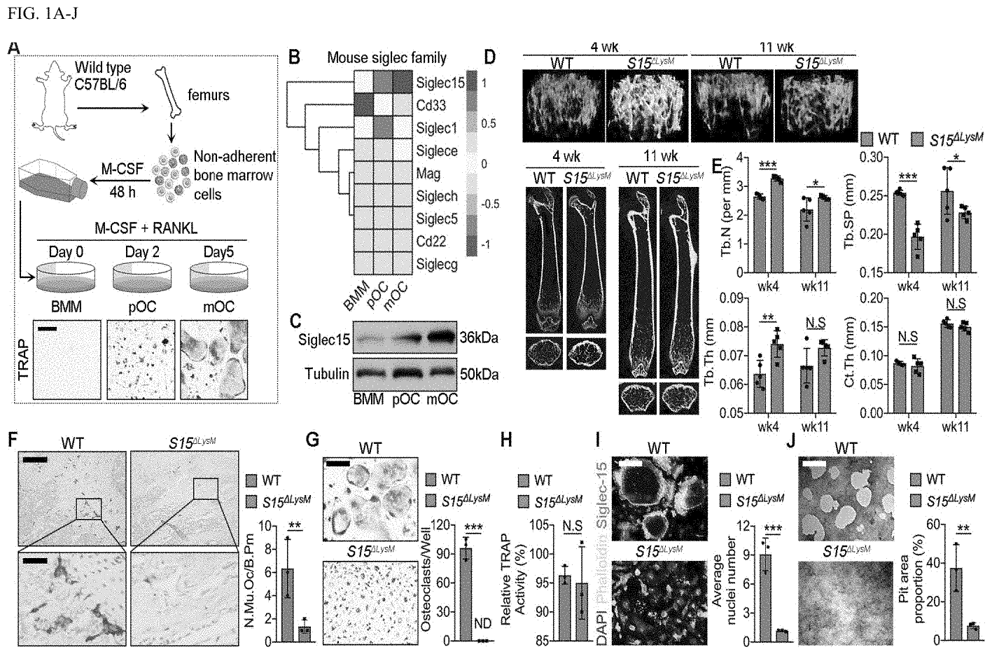

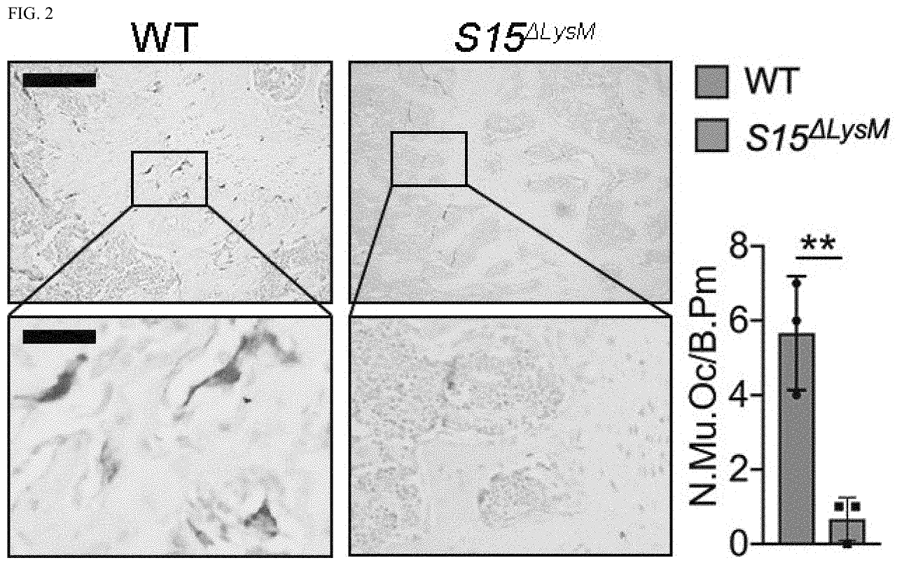

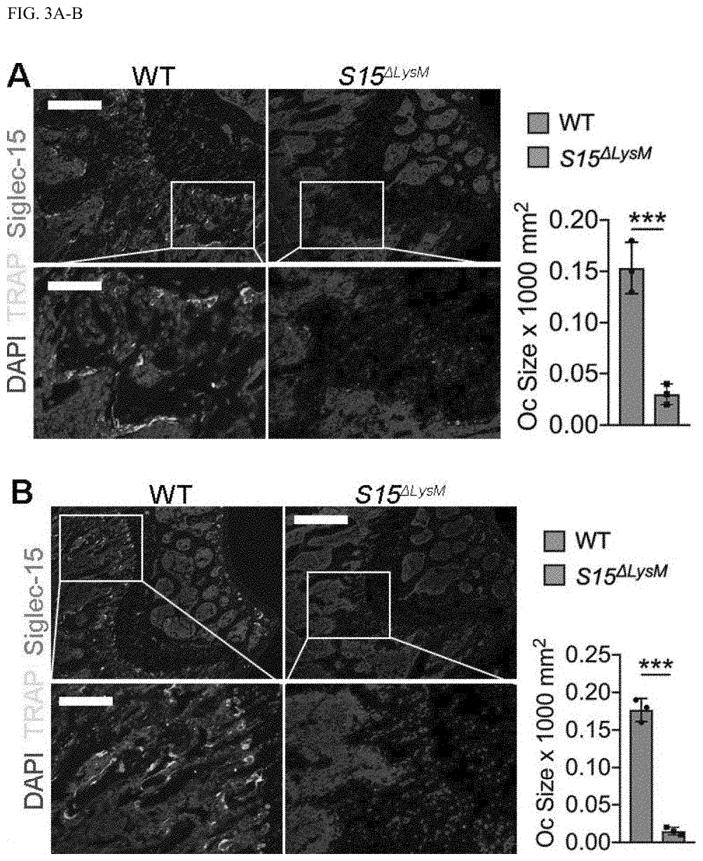

We first isolated the whole bone-marrow cells from C57BL/6 mice hind limbs and stimulated them with M-CSF (50 ng/mL) for 48 h to acquire bone marrow macrophages (BMMs). BMMs were then treated with M-CSF (30 ng/mL) and RANKL (100 ng/mL) for 48 h for pre-osteoclasts (pOCs) and 120 h for mature osteoclasts (mOCs) (FIG. 1A). Next, we performed an unbiased global transcriptomic comparison of BMM, pOC, and mOC by RNA sequencing (RNA-seq). The expression profile of the total mouse Siglec family was clustered, showing that Siglec15, which is conserved in most mammals, including humans, is the only continuously upregulated Siglec during osteoclast differentiation (FIG. 1B). The upregulation of Siglec15 was validated by western blotting on the protein level (FIG. 1C). To determine the role of osteoclast-associated Siglec15 in vivo, we crossed Siglec15^{ﬂ/ﬂ} mice with the LysM-Cre strain to produce a deletion of Siglec15 on macrophage/granulocytes (Siglec15^{ΔLysM}) (Wang et al., 2019). Siglec15 is found only on macrophages, not granulocytes; therefore, this strain provides a unique tool to study the function of Siglec15 in BMMs, pOCs, and mOCs. We then used μCT to evaluate the bone volume change of young (4-week-old) and adult (11-week-old) Siglec15^{ΔLysM} mice relative to that of Siglec15^{ﬂ/ﬂ} littermates (FIG. 1D). Consistent with previous studies using Siglec15 global knockout mice (Hiruma et al., 2013; Kameda et al., 2013), our results suggested an increase in trabecular bone volume in young and adult Siglec15^{ΔLysM} mice, marked by significantly increased trabecular thickness (Tb. Th) and trabecular number (Tb. N), as well as significantly decreased trabecular separation (Tb. Sp). Cortical thickness (Ct. Th) showed no significant change (FIG. 1E). TRAP staining results of distal femur sections showed that in young and adult Siglec15^{ΔLysM} mice, the number of multinucleated osteoclasts was much lower, and osteoclasts were small and did not spread on the bone (FIG. 1F, 2). Immunofluorescent staining of Siglec15 and TRAP confirmed that Siglec15 and TRAP levels decreased robustly in young and adult Siglec5^{ΔLysM} mice distal femurs (FIG. 3).

To further investigate the role of Siglec15 in osteoclast formation, we isolated and cultured BMMs from Siglec15^{ΔLysM} mice and stimulated cells with M-CSF and RANKL. TRAP staining results showed that on day 5, large multinucleated osteoclasts were formed in the wild-type (WT) group but were not detected in the Siglec15^{ΔLysM} group (FIG. 1G). However, relative TRAP activity results showed no significant difference between the 2 groups (FIG. 1H). Immunofluorescent staining of Siglec15 and phalloidin showed that Siglec15-deficient BMMs failed to form multinucleated osteoclasts with a complete actin ring (FIG. 1I). Pit formation assay showed a robust decrease in the bone resorption activity of Siglec15-deficient osteoclasts, marked by the significantly decreased pit area proportion by the significantly decreased pit area proportion (FIG. 1J).

### Example II

This example demonstrates that Siglec15 expression in osteoclasts is induced by M-CSF via MEK-ERK-MYC signaling.

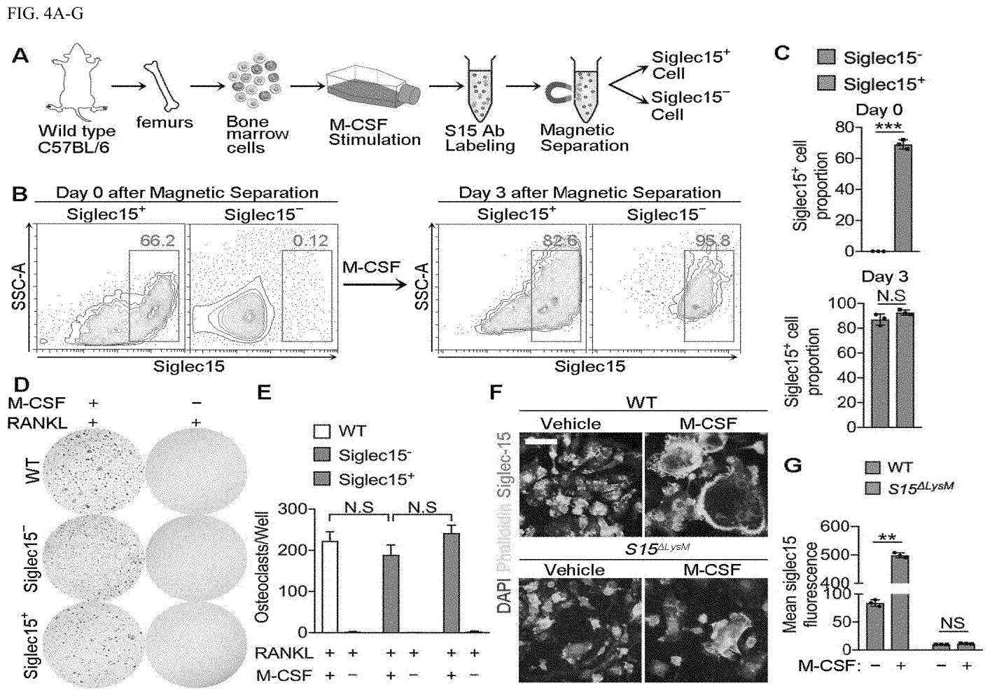

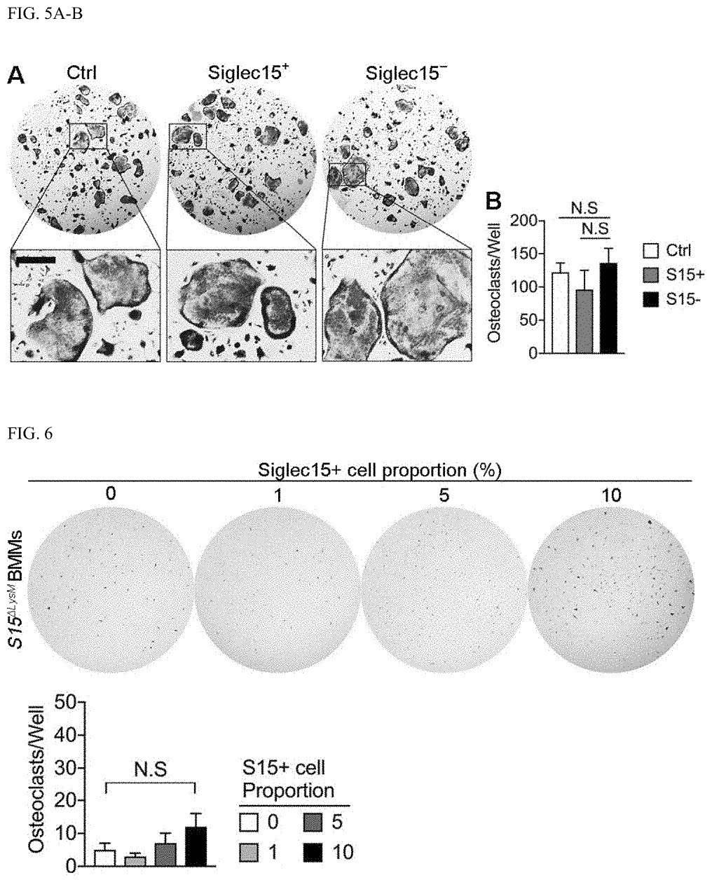

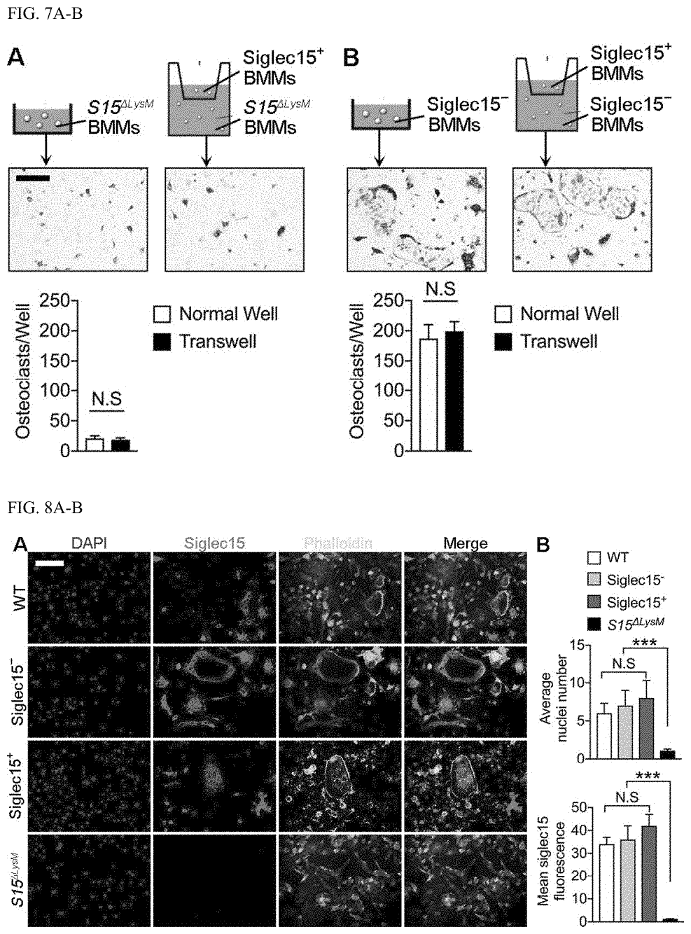

To understand the detailed mechanism of Siglec15 regulating osteoclast formation, we incubated WT BMMs with biotinylated Siglec15 monoclonal antibody and manually separated Siglec15⁺ BMMs from Siglec15- BMMs using anti-biotin microbeads and magnetic separators (FIG. 4A). Separated Siglec15* and Siglec15- BMMs were then stimulated with M-CSF and RANKL for osteoclastogenesis. Surprisingly, no significant difference was observed in osteoclast formation capacity between Siglec15⁺ and Siglec15-BMMs (FIG. 5). One study reported that osteoclast fusion might be initiated by a small subset of progenitors (Levaot et al., 2015); therefore, we combined separated Siglec15⁺ BMMs with BMMs isolated from Siglec15^{ΔLysM} mice in different proportions (1%, 5%, and 10%). TRAP staining results showed that Siglec15⁺ BMMs failed to initiate osteoclast fusion in BMMs deficient of Siglec15 (FIG. 6). Furthermore, transwell culture showed that Siglec15⁺ BMMs also have no indirect effects on osteoclastogenesis of WT, Siglec15⁻, and Siglec15^{-/-} BMMs (FIG. 7). Immunofluorescent staining confirmed that Siglec15⁺ and Siglec15⁻ BMMs can form multinucleated osteoclasts with normal actin rings, and the expression of Siglec15 was restored in Siglec15⁻ BMMs during osteoclastogenesis (FIG. 8). Separated Siglec15⁻ BMMs were then cultured with M-CSF, and FCM analysis showed that Siglec15⁻ BMMs restored Siglec15 expression after 3 days of stimulation (FIG. 4B, C). BMMs cultured with RANKL alone, absent M-CSF, cannot survive during osteoclastogenesis (FIG. 4D, E).

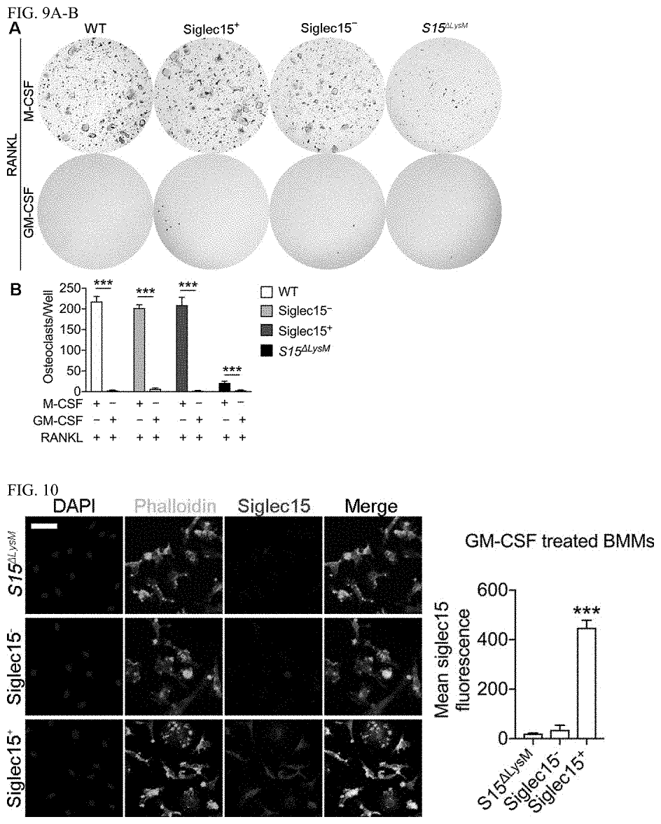

Immunofluorescent staining results showed that M-CSF stimulation significantly increased the proportion of Siglec15⁺ BMMs in Siglec15^{ﬂ/ﬂ} mice (FIG. 4F, G). We then tested the other CSF family member, granulocyte-macrophage CSF (GM-CSF, encoded by CSF2), and found that GM-CSF cannot induce osteoclastogenesis (FIG. 9) nor the expression of Siglec15 (FIG. 10).

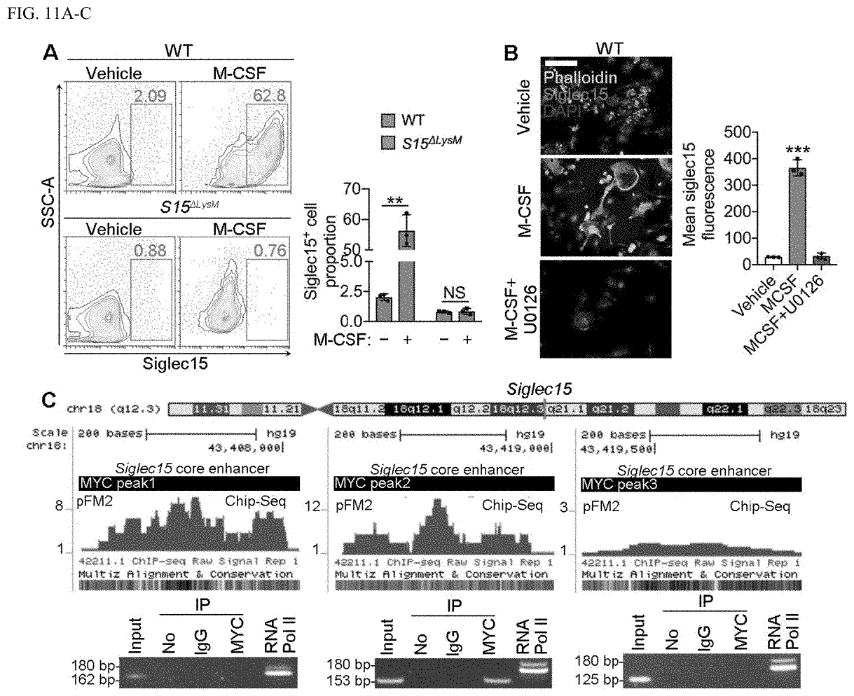

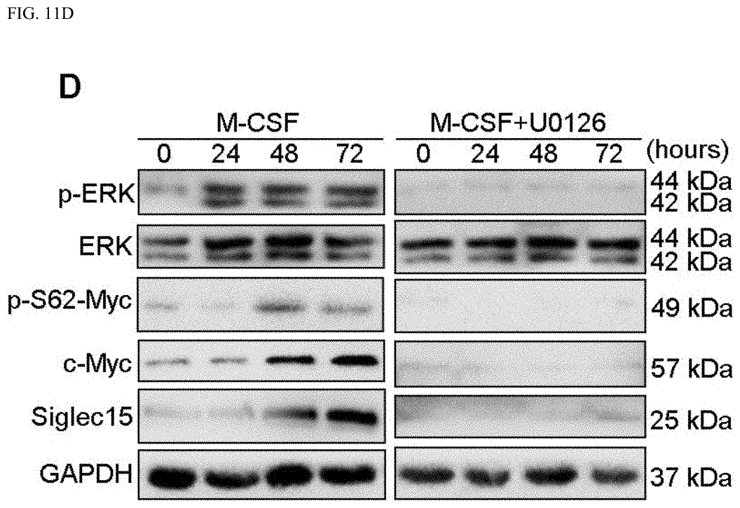

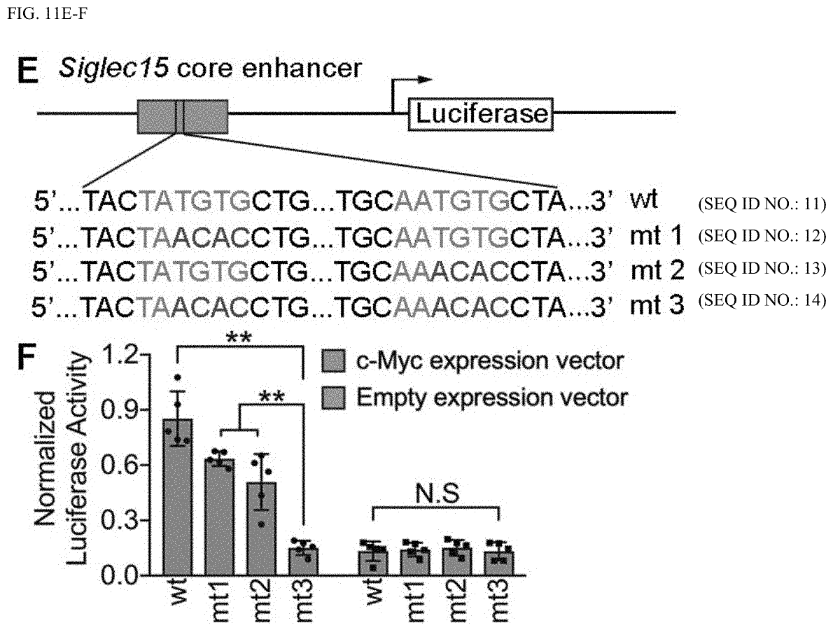

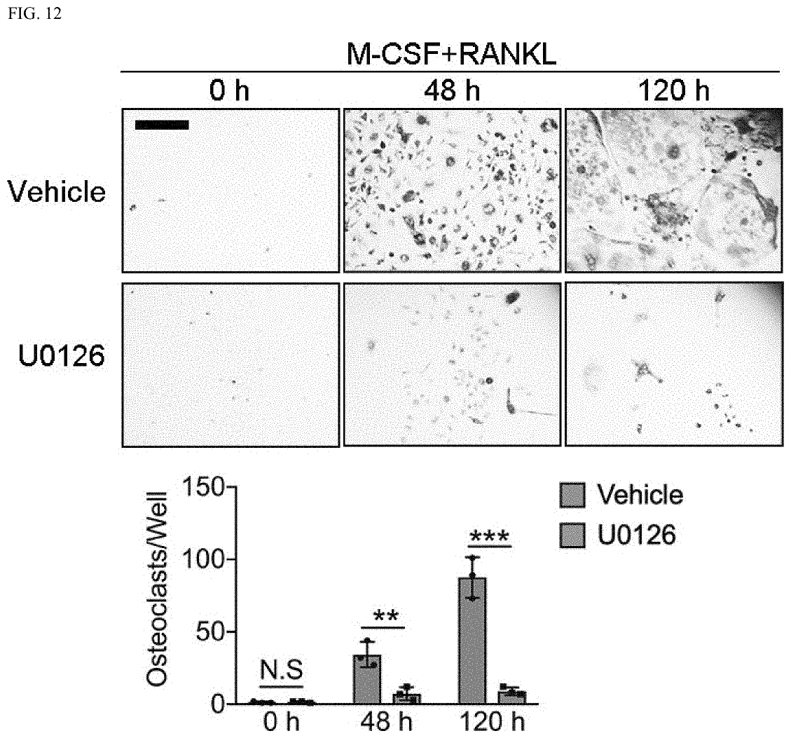

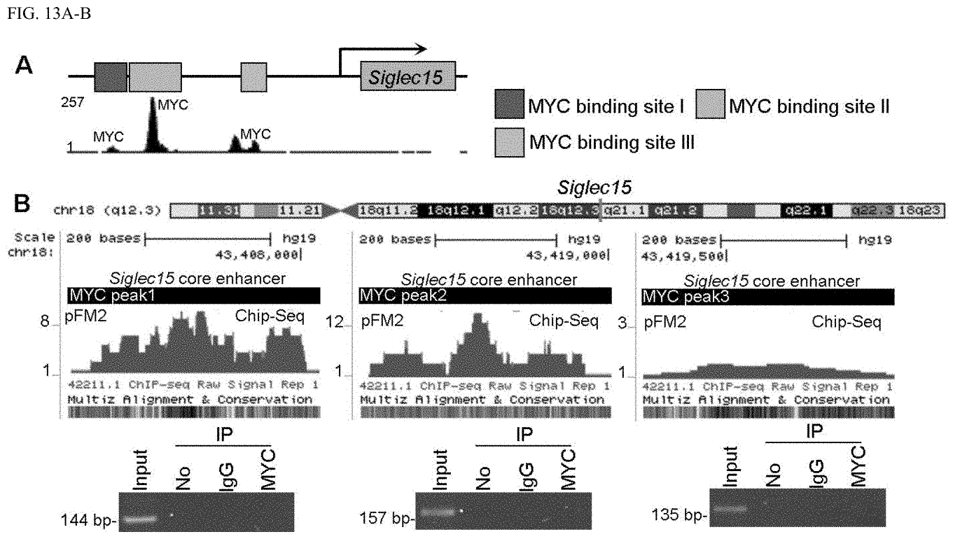

Using flow cytometry (FCM), we confirmed that in vitro M-CSF stimulation of whole bone-marrow cells significantly increased the proportion of Siglec15⁺ BMMs in Siglec15^{ﬂ/ﬂ} mice, whereas Siglec15^{ΔLysM} mice showed no obvious changes (FIG. 11A). To explore the molecular mechanism of M-CSF activation of Siglec15, we used U0126, a selective of mitogen-activated protein kinase kinases inhibitor of MEK1 and MEK2. BMMs treated with U0126 showed decreases in osteoclastogenesis and cell survival rate (FIG. 12). Siglec15 upregulated by M-CSF was also abrogated by U0126 treatment (FIG. 11B). To understand Siglec15 transcription, we analyzed the core enhancer of Siglec15 and found three binding sites of MYC with high species controversy. Chromatin immunoprecipitation (ChIP) assay was performed to investigate the interaction of MYC with the 3 deoxyribonucleic acid binding sites. Electrophoresis of the polymerase chain reaction (PCR) amplification products showed that MYC binds with a 310-bp region at the core enhancer of Siglec15 (FIG. 11C). Western blot results confirmed that M-CSF-mediated extracellular signal-regulated kinase (ERK) activation phosphorylated the Ser62 of MYC, thus stabilizing the MYC to further activate Siglec15 expression. U0126 treatment blocked this cascade from the inhibition of ERK activation (FIG. 11D). ChIP-PCR results showed that GM-CSF cannot induce the binding of MYC with the core enhancer of Siglec15 (FIG. 13). MYC recognizes and binds to E-box domains with consensus recognition sequence of CACGTG or CATGTG (Guccione et al., 2006). In the 310-bp binding region, we recognized the sequences of TATGTG and AATGTG that share 80% similarities with reported binding bases. To confirm the role of these MYC recognition sequences, we constructed an enhancer reporter with this Siglec15 enhancer, and 3 mutants were derived by site-directed mutagenesis to inactivate the MYC recognition (FIG. 11E). Enhancer analysis showed that these MYC recognition sequences are responsible for MYC-dependent activation of Siglec15 (FIG. 11F). Thus, M-CSF activates Siglec15 expression via MEK-ERK-MYC cascade.

### Example III

This example demonstrates that sialylated TLR2 is the binding ligand for Siglec15.

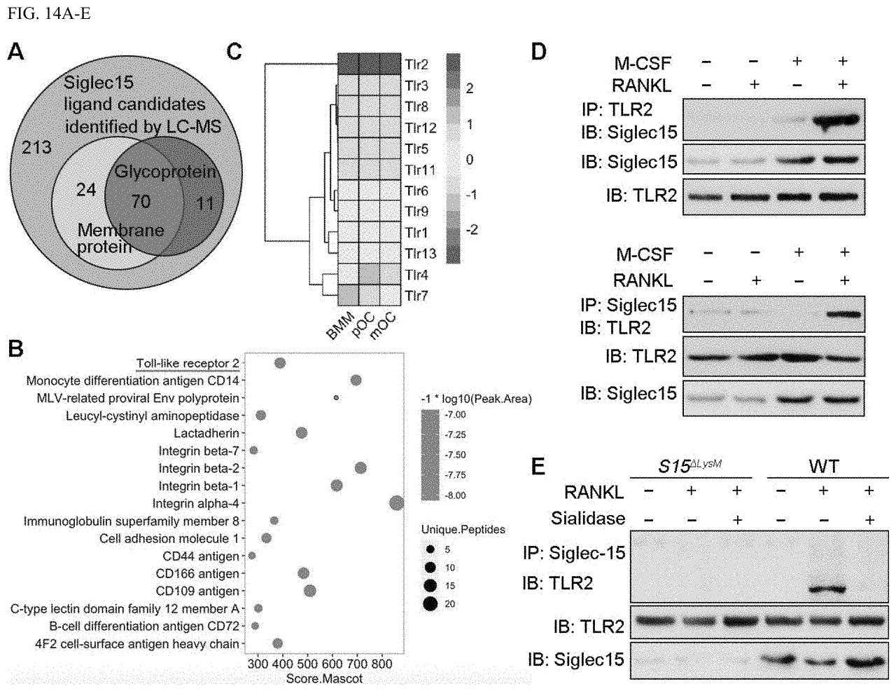

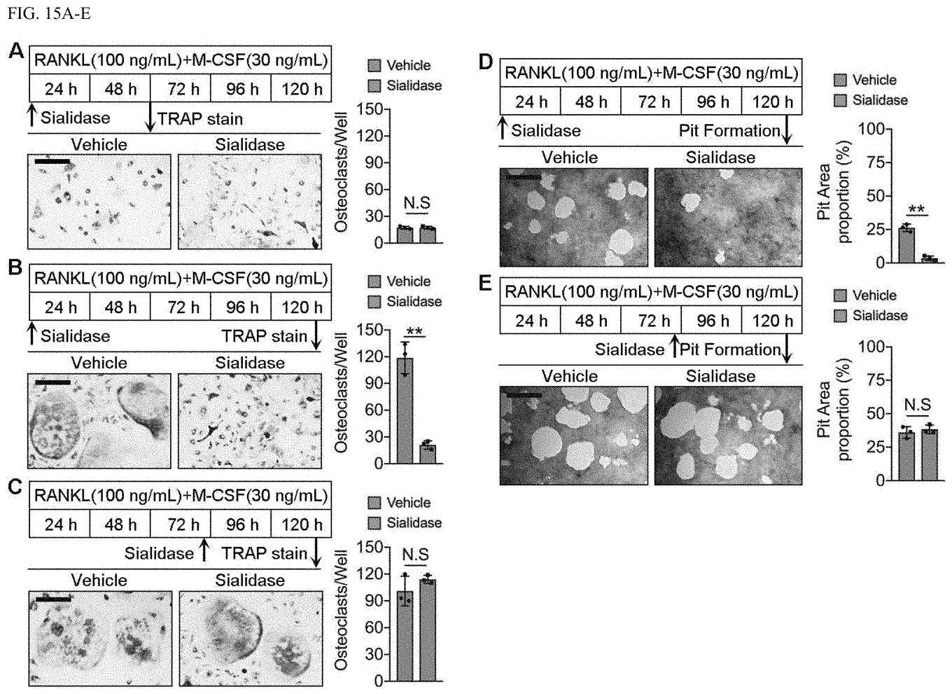

To determine the ligands of Siglec15 in osteoclastogenesis, we adopted a liquid chromatography-mass spectrometry dataset (ProteomeXchange Consortium, PXD006359) identifying Siglec15 interacting proteins using proximity labeling methods (Chang et al., 2017). The results showed that 318 proteins are potential candidates for Siglec15 ligands. The candidate number decreased to 70 after narrowing the scope to membrane glycoprotein (FIG. 14A). TLR2 is screened out for the highest overall score of unique peptides, peak area, and Mascot score (FIG. 14B). Using RNA-seq, we also found that the expression of TLR2 is at a consistently high level during osteoclastogenesis among its family members (FIG. 14C). The interaction of Siglec15 and TLR2 was verified by co-immunoprecipitation (IP) in WT BMM-induced osteoclasts (FIG. 14D). Siglec15-TLR2 interaction could not be detected in BMMs isolated from Siglec15^{ΔLysM} mice. Furthermore, sialidase treatment disrupted the formation of the Siglec15-TLR2 complex in WT osteoclasts, suggesting that sialylation of TLR2 is essential for the Siglec15-TLR2 complex formation (FIG. 14E). To test the effects of sialidase disruption on the Siglec15-TLR2 interaction in osteoclast differentiation, we treated WT BMMs with sialidase at different time points. Sialidase treatment did not affect the formation of TRAP⁺ pOCs or the bone resorption activity of mOCs; however, sialidase treatment did block the fusion of pOCs into multinucleated mOCs (FIG. 15).

### Example IV

This example demonstrates that RANKL-induced ST3Gal1 transcription by activation binding of FOS to its promoter, and that sialyltransferases ST3Gal1 and ST3Gal4 induce sialylation modification of TLR2/4 as therapeutic target for cancer and bone diseases.

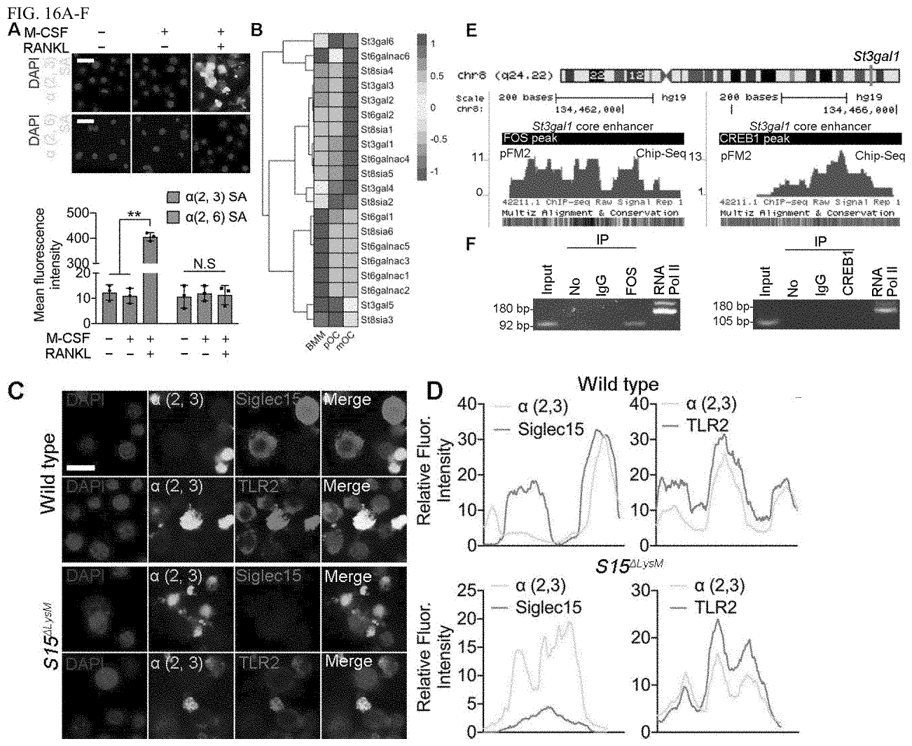

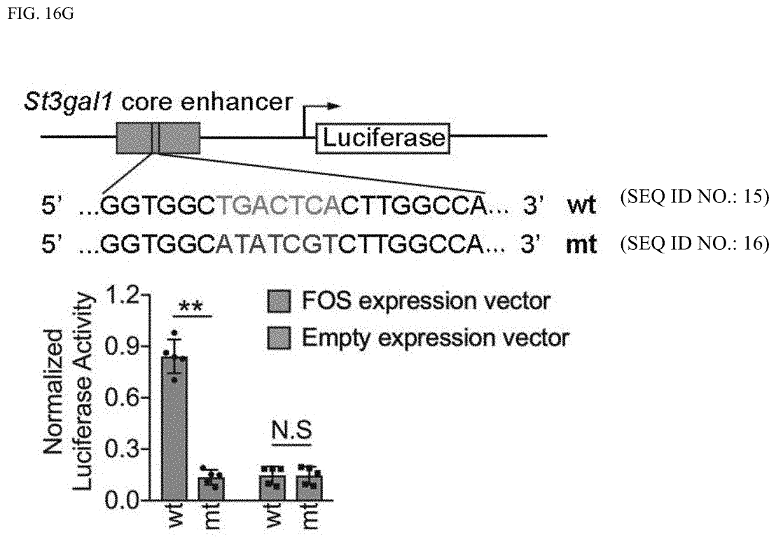

Because TLR2 has 4 N-linked glycosylation sites (Weber et al., 2004), to investigate the molecular mechanism of sialylation we tested α2,3 and α2,6 N-Linked SA modification, the two most common sialylation pattern in mammalian cells. We found that RANKL, not M-CSF, robustly induced α2,3 sialylation of TLR2 in WT analysis of BMMs (FIG. 16A). To understand why α2,3 was sialylated, we examined each member expression profile of the entire sialyltransferase family. The RNA-seq data showed that among STs that express in skeletal tissue, ST3Gal1, which encodes enzyme catalyzing α2,3 sialylation, was significantly upregulated, whereas ST6Gal1, which encodes enzyme catalyzing α2,6 sialylation, was downregulated (FIG. 16B). Moreover, α2,3 SA was immunocolocalized with TLR2, not with Siglec15 in WT and Siglec15^{ΔLysM} BMMs after RANKL stimulation for 24 h (FIGS. 16C, D).

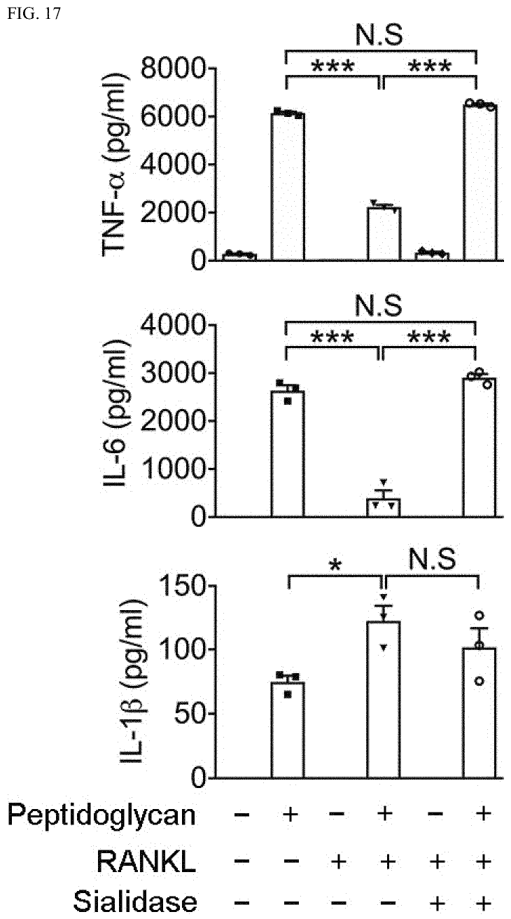

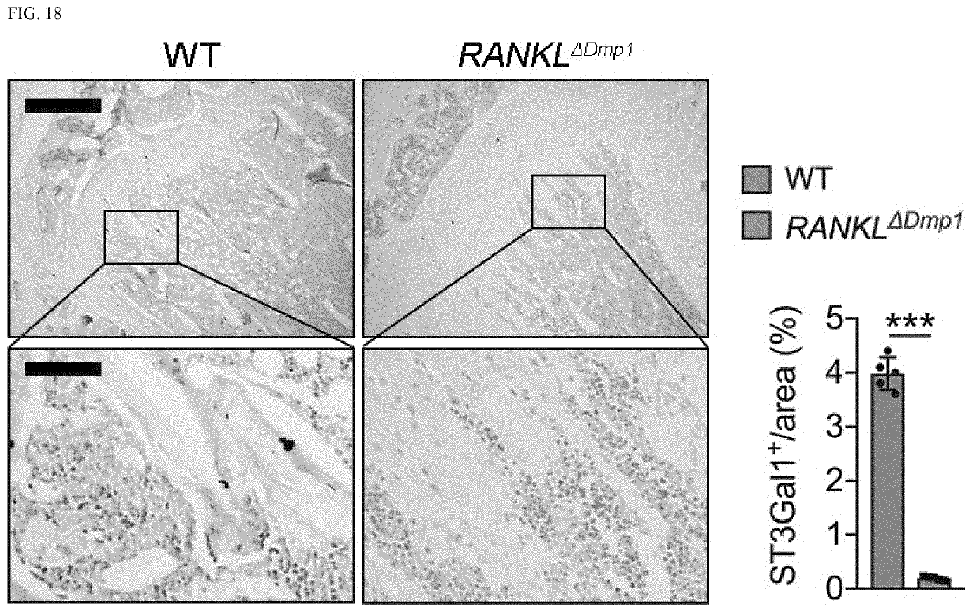

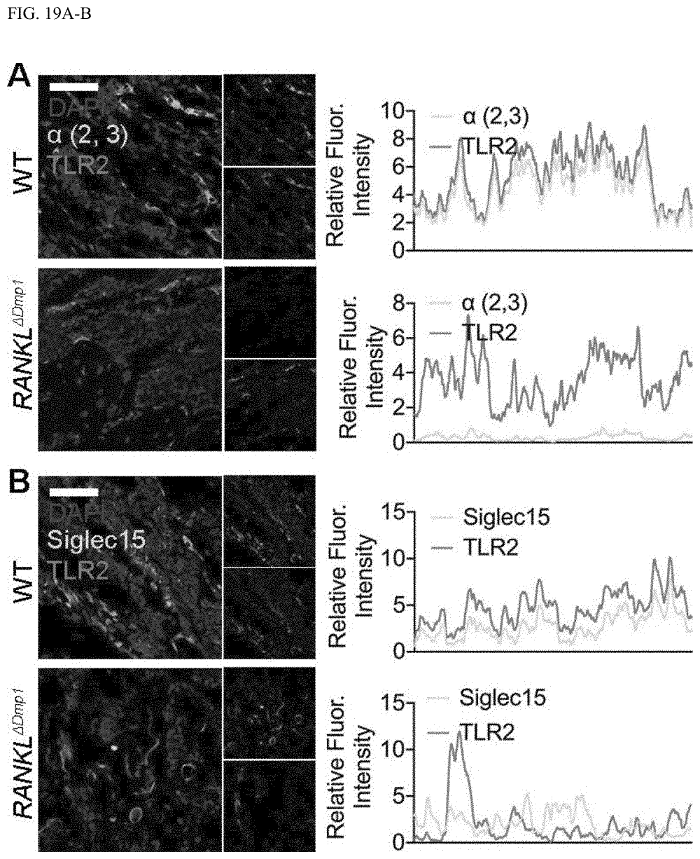

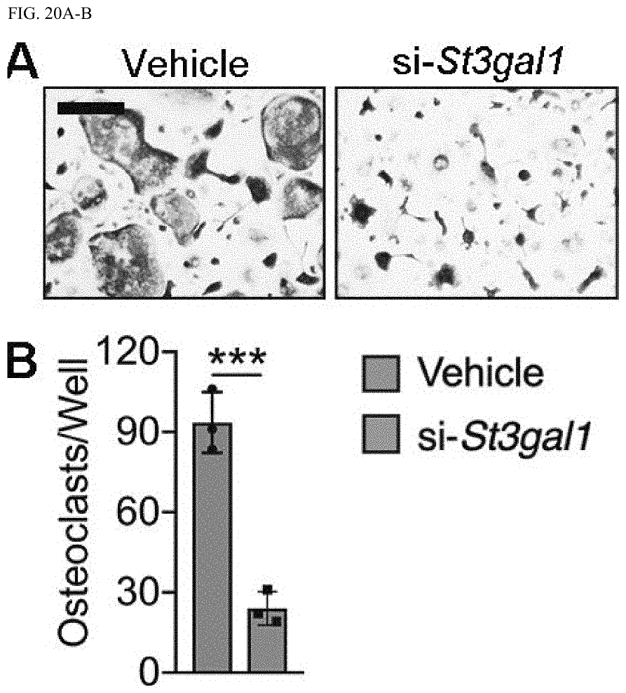

To test signaling activity of α2,3 sialylation, we removed α2,3 SA of TLR2 induced by RANKL by adding sialidase in the BMM culture. Enzyme-linked immunosorbent assay showed that peptidoglycan-induced secretion of Tumor necrosis factor-alpha (TNF-α) and Interleukin 6 (IL-6) from BMMs through activation of TLR2 was inhibited by RANKL, and the addition of sialidase restored TLR2 activity and TNF-α and IL-6 levels (FIG. 17). To examine the transcriptional mechanism of ST3Gal1 activation by RANKL, we analyzed 5′ promoter sequence of ST3Gal1 and recognized binding sites for CREB1 and FOS (downstream transcription factors of RANKL signaling) in the core enhancer (FIG. 16E). ChIP-PCR assay showed that FOS but not CREB1 bound to the core enhancer of ST3Gal1 (FIG. 16F). To confirm the functional activity of the FOS site, we constructed an enhancer reporter with ST3Gal1 enhancer and FOS mutant binding sites. Transcriptional activity analysis showed that the FOS recognition sequence TGACTCA is responsible for FOS-dependent activation of ST3Gal1 (FIG. 16G). To validate RANKL-induced sialylation in vivo, we crossed RANKL^{ﬂ/ﬂ} mice with the Dmp1-Cre strain to generate osteocyte-specific RANKL knockout mice (RANKLΔ^{Dmp1}). Indeed, immunohistostaining showed that expression of ST3Gal1 in the distal femur of RANKL^{ΔDmp1} mice significantly decreased relative to RANKL^{ﬂ/ﬂ} mice (FIG. 18). Furthermore, α2,3 SA levels were significantly downregulated in RANKL^{ΔDmp1} mice relative to RANKL^{ﬂ/ﬂ} mice (FIG. 19A). Furthermore, the Siglec15-TLR2 co-localization that can be observed in RANKL^{ﬂ/ﬂ} mice disappeared in RANKL^{ΔDmp1} mice (FIG. 19B). In vitro study confirmed that St3gal1 siRNA transfection disturbed osteoclast fusion marked by a robust reduction in the number of osteoclasts (FIG. 20).

It was also found that, in rheumatoid arthritis, endogenous ST3Gal4, but not ST3Gal1 is significantly increased, whereas in ankylosing spondylitis, both ST3Gal4 and ST3Gal1 expression levels are increased. The elevated expression of sialic acid transferase is specific in different disease. Therefore, inhibition of ST3Gal1 could be an effective therapeutic target. For example, inhibition of sialic acid transferase ST3Gal1 activity could be target for cancer and osteoporosis, inhibition of ST3Gal4 for rheumatoid arthritis, inhibition of both ST3Gal4 and ST3Gal1 for ankylosing spondylitis.

### Example V

This example demonstrates that injection of sialidase reduced osteoclast formation and bone remodeling in Siglec15^{ﬂ/ﬂ} mice, but such effect was abrogated in Siglec15^{ΔLysM} mice.

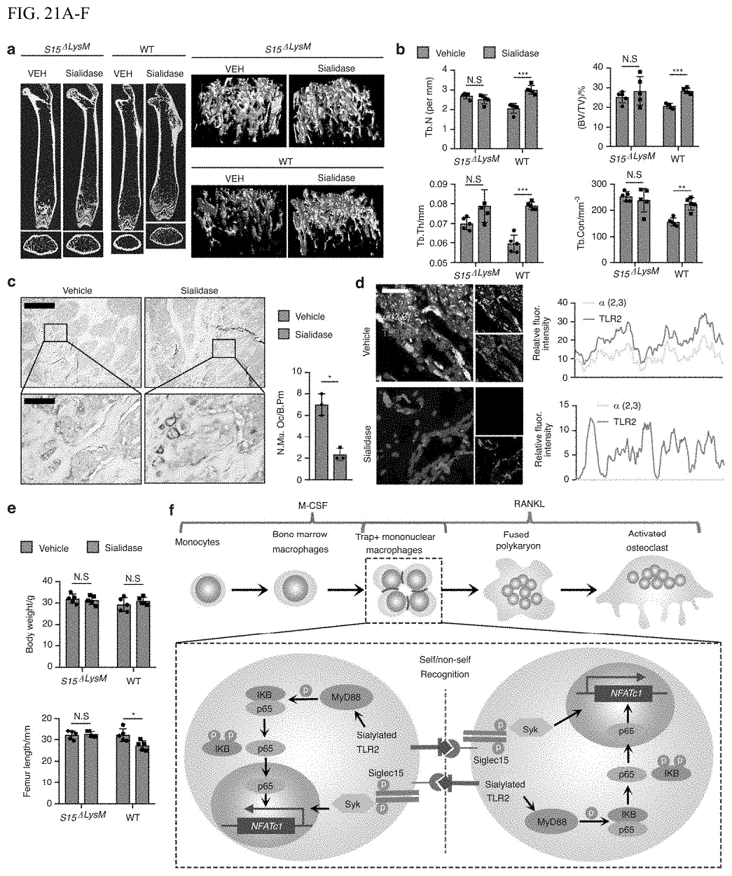

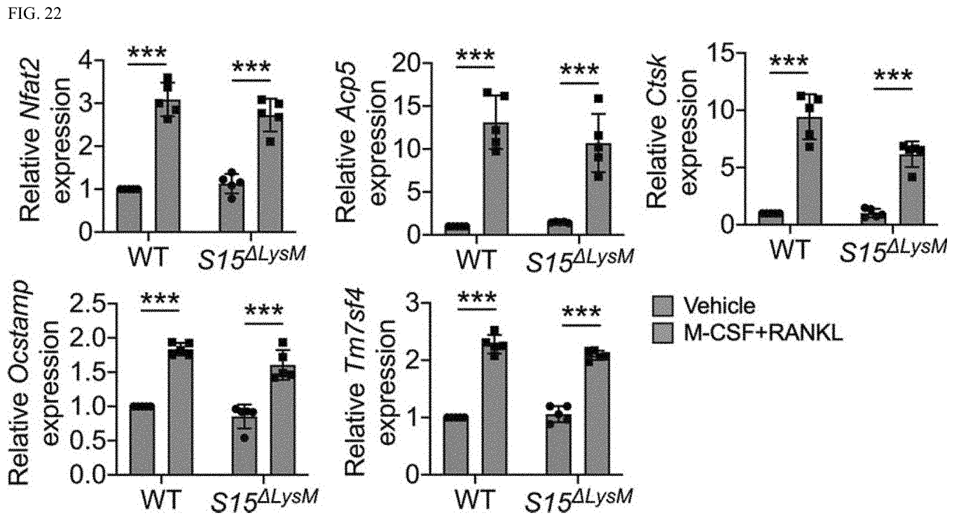

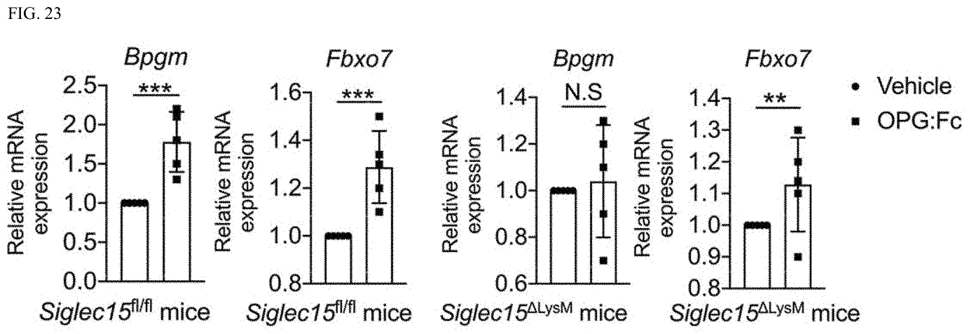

To validate that sialylation of TLR2 originates with binding to Siglec15 for osteoclast fusion, we injected sialidase intrafemorally into Siglec15^{ΔLysM} mice and their Siglec15^{ﬂ/ﬂ} littermates. After 4 weeks of injections, the mice were euthanized, and the femurs were collected for μCT scanning and histological analysis (FIG. 21a). μCT analysis indicated that trabecular bone volume (TV/BV), Tb. Th, Tb. N, and trabecular connectivity (Tb. Con) were significantly increased in Siglec15^{ﬂ/ﬂ} mice injected with sialidase (FIG. 21b), indicating a decrease in osteoclast-induced bone remodeling. Indeed, TRAP staining of distal femur sections showed that the number and size of multinucleated osteoclasts in the sialidase-treated mice were decreased significantly relative to those in control mice (FIG. 21c). The number of osteoclast precursors on the bone surface was slightly higher but not significantly different in Siglec15^{ΔLysM} mice treated with sialidase (FIG. 22). Further coimmunofluorescence staining for α2,3 SA and TRAP revealed that sialylation was abrogated in mouse distal femurs injected with sialidase (FIG. 21d), and again, no changes were observed in Siglec15^{ΔLysM} mice. The femurs of Siglec15^{f}/f mice injected with sialidase were shorter than those of vehicle-injected mice, suggesting a delay in femur growth, whereas no difference was observed between Siglec15^{ΔLysM} mice injected with sialidase or vehicle (FIG. 21e). A recent report showed that osteoclasts are recycled via fission into daughter osteomorphs (see, McDonald, 2021). By testing the expression of Bpgm and Fbxo7 in the bone marrow of OPG:Fc-treated WT and Siglec15^{ΔLysM} mice, a potential downregulation of the osteomorph reservoir was observed in Siglec15^{ΔLysM} mice (FIG. 23), suggesting an inhibition of multinucleated osteoclast formation. Our results show that the sialylation of TLR2 initiates its interaction with Siglec15 to induce cell fusion for osteoclast formation, bone remodeling, and bone growth.

### Example VI

This example provides a discussion of Examples I-V.

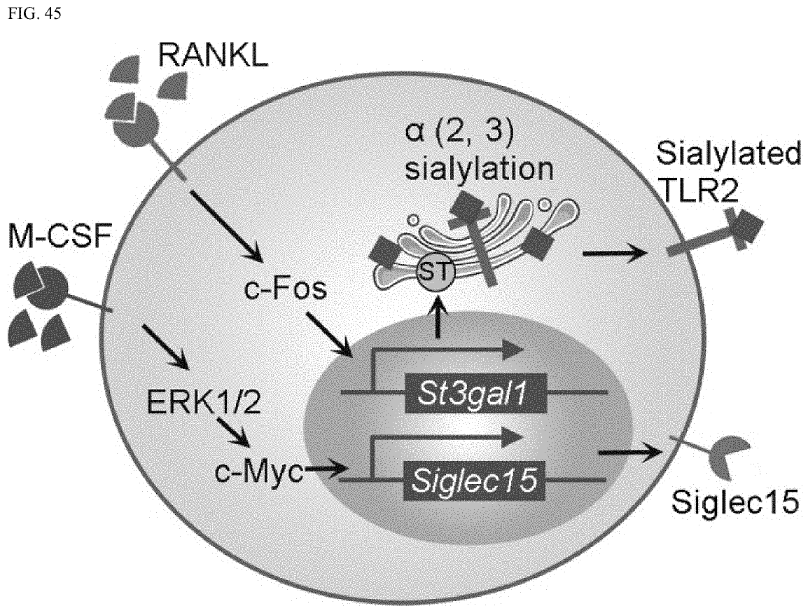

Many skeletal disorders are involved in aberrant osteoclast differentiation including skeleton metastasis. Currently, we still have not be able to develop effective therapy for these skeletal diseases, largely due to limited knowledge of preosteoclast fusion for formation of multinucleated osteoclasts. It is quite clear that two factors are critical for osteoclast differentiation: M-CSF for progenitor cell survival and proliferation(Ross and Teitelbaum, 2005) and RANKL for their for the commitment to osteoclast lineage differentiation (Boyle et al., 2003). In this study, we found the cell-recognition signal of Siglec15-TLR2 interaction initiates fusion of mononuclear cells discriminating self from non-self. Interestingly, we showed that Siglec15 expression is activated by M-CSF while TLR2 sialylation is induced by RANKL (FIG. 45), indicating that both M-CSF and RANKL are required for the formation of the recognition signal for osteoclast fusion (FIG. 21). The Siglec15 has been reported in regulation of osteoclast differentiation by association with DNAX-activating protein 12 kDa (DAP12) upon activation by unknown ligands (Hiruma et al., 2011; Kameda et al., 2013; Stuible et al., 2014). We now show that α2,3 sialylated TLR2 is the ligand for Siglec15 as the signal of fusion of mononuclear cells. A recent cell-based glycan array study also detected that α2,3 SA has specific high affinity with Siglec15 (Briard et al., 2018). Mononuclear cells prior to fusion exhibit distinct anabolic function, whereas fused multinucleated osteoclasts are catabolic in resorption of bone. Trap+ mononuclear cells maintain on the periosteal surface for cortical bone formation (Baroukh et al., 2000; Ochareon and Herring, 2011). Mononuclear cells have been observed in aquatic vertebrate skeleton, where there is continuous growth with no osteoclast bone resorption (Chatani et al., 2011; Witten and Huysseune, 2009). The well-controlled regulation between mononuclear cells and fused multinucleated osteoclasts balances bone anabolic or catabolic activity. Shift the balance toward osteoclast formation lead to bone loss such as postmenopausal osteoporosis and breast cancer metastasis in bone. Our finding of the control signal between mononuclear cells and osteoclasts implicates the potential therapeutic target for these skeletal diseases.

We previously have shown that mononuclear cells secrete platelet-derived growth factor (PDGF)-BB for type H blood vessel formation in coupling osteogenesis (Xie et al., 2014). Mononuclear cells controls the coupling between osteogenesis and angiogenesis during bone remodeling or modeling. In postmenopausal osteoporosis, mononuclear cells are fused to osteoclasts much earlier with a relative shorter lifespan (Manolagas, 2000; Pacifici, 1996). As a result, the osteoclastic bone resorption is increased whereas blood vessel formation in supporting bone formation is decreased, the net outcome is catabolic bone loss. Hence, the signaling mechanism that triggers fusion of mononuclear cells is critical in maintaining bone homeostasis and developing potential therapies for those immedicable skeletal disorders. It is interesting to notice that TLR activation in BMMs abolished their differentiation to osteoclasts (Krisher and Bar-Shavit, 2014; Takami et al., 2002). Our results showed that TLRs on BMMs were modified with α2,3 glycans stimulated by RANKL as a ligand for Siglec15. The recognition and binding between Siglec15 and sialylated TLR2 activates the Siglec15-associated DAP12 that co-stimulates osteoclast differentiation (Koga et al., 2004) and also blocks the TLR signaling that inhibits osteoclast differentiation. It has been shown that sialylation of cell surface glycoconjugates is essential for osteoclastogenesis (Takahata et al., 2007). Importantly, we demonstrate that inhibition of sialylation blocks TLR signaling for osteoclast fusion and differentiation. Binding of TLRs to Siglec15 may not be limited to TLR2 as interactions between TLRs and Siglec families have been broadly detected (Chen et al., 2014). For example, It has been reported that Siglec-E binding with TLR4 negatively regulates its activation (Wu et al., 2016) and TLR induced expression of SOCS1 and SOCS3 is reduced in Siglec2-deficient B cells (Kawasaki et al., 2011).

Autoimmune disease is often associated with imbalanced bone remodeling of increased bone resorption, such as rheumatoid arthritis (RA) and systemic lupus erythematosus (SLE) arthritis. In RA, excessive RANKL was produced by synovial fibroblasts stimulated by T_{H}17 derived IL-17 (Sato et al., 2006). In SLE patients, soluble RANKL level was significantly increased in the serum (Carmona-Fernandes et al., 2011). The upregulated RANKL in these autoimmune diseases accelerates fusion of mononuclear cells. As a result, the lifespan of mononuclear cells is shortened with increase of osteoclast maturation for bone degradation, which explains most of autoimmune related bone disorders with bone destruction. Indeed, serum sialic acid levels were increased in RA patients with potential use as biomarker for prediction and severity of RA in clinical practice (Alturfan et al., 2007; Li et al., 2019). In both SLE and RA patients, ST3Gal1/Neu3 ratio was found positively correlated with the disease activity (Liou and Huang, 2016). Similarly in cancer, aberrant sialylation has long been associated with metastatic cell behaviors including invasion to bone (Schultz et al., 2012). Prostate cancer has the highest spread rate to bones (Hemandez et al., 2018) and elevated SA level is an independent predictor of prostate cancer and its bone metastases (Zhang et al., 2019). Metabolomics study also showed that SA plays key role in breast cancer metastasis (Teoh et al., 2018).

In summary, the described findings of sialylation of TLR2 as cell-fusion signal controls the balance between mononuclear cells and osteoclasts for bone homeostasis and explains bone destruction in autoimmune disease and cancer metastasis in bone.

### Example VII

This example demonstrates that binding of Siglec15 and TLR2 activates both of their downstream signaling pathways.

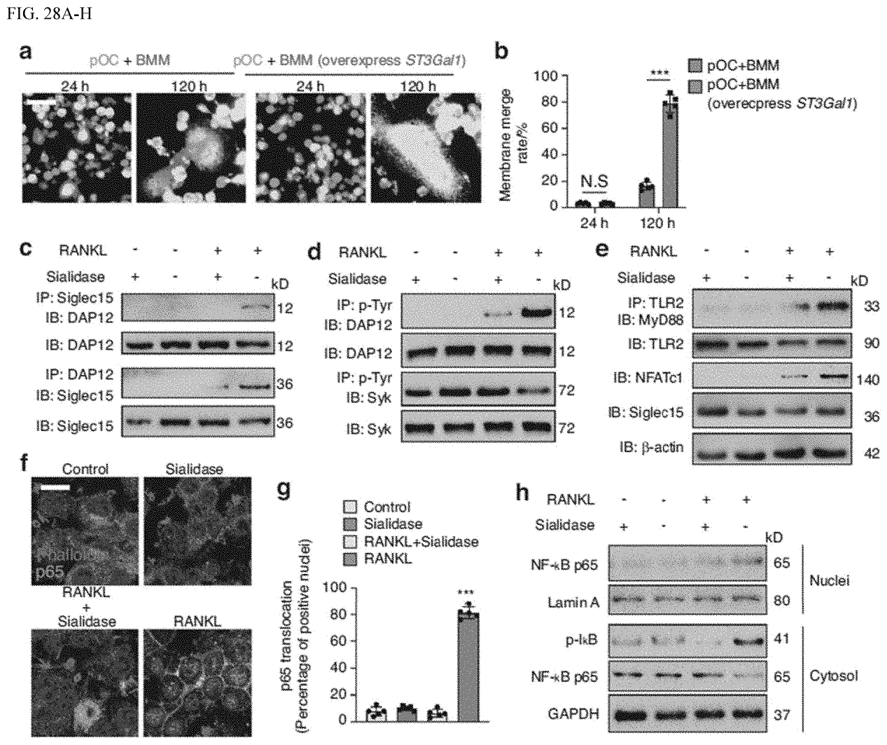

Siglec15 deficiency led to smaller OCs that were still TRAP+, so we further tested the gene expression of the osteoclastogenesis master regulator Nfat2 (NFATc1), osteoclastic marker Acp5 (TRAP), ctsk, and the fusogenic genes ocstamp and Tm7sf4 (DC-STAMP) in WT and Siglec15^{ΔLysM} BMMs using qPCR. The results showed that upon M-CSF+ RANKL stimulation, the expression of Nfat2, Acp5, ctsk, oc-stamp, and Tm7sf4 was not affected by Siglec15 deficiency (FIG. 45). These results suggest that the process of cell recognition mediated by Siglec15 binding to its receptor sialylated TLR2 occurs prior to cell fusion. We assumed that the cell recognition patterned by Siglec15-TLR2 binding enables Trap+ macrophages to distinguish themselves from nonself cells and serves as a prerequisite for cell fusion. For validation, Trap+ mononuclear cells were cocultured with BMMs, and no obvious fusion was detected; however, Trap+ mononuclear cells cocultured with BMMs overexpressing ST3Gal1 showed a significantly higher membrane fusion rate with observable fused multinucleated osteoclasts (FIG. 28a, b), indicating that binding between Siglec15 and sialylated TLR2 initiates cell fusion. In particular, the cell recognition mediated by Siglec15 binding to its receptor sialylated TLR2 suggests activation of downstream signaling by both Siglec15 and sialylated TLR2. We therefore examined whether the binding between Siglec15 and sialylated TLR2 activates bidirectional signaling in Trap+ mononuclear cells during the initiation of cell fusion.

To examine whether Siglec15 downstream signaling is activated by binding to sialylated TLR2, co-IP of Siglec15 with DAP12, a key factor for cell fusion, was conducted in BMMs induced with RANKL, sialidase or both. RANKL-induced the association of Siglec15 with DAP12, while sialidase impaired the association (FIG. 28c). Moreover, tyrosine phosphorylation of DAP12 was detected in the co-IP experiment upon RANKL induction of Siglec15 clustering but was again impaired by sialidase treatment (FIG. 28d). We then tested whether TLR2 signaling is activated by binding to Siglec15. The recruitment of MyD88 to TLR2 was examined by co-IP. Binding of Siglec15 induced TLR2 association with MyD88 and resulted in upregulation of NFATc1. Removal of SA by sialidase reduced MyD88 recruitment and NFATc1 activation (FIG. 28e). Immunostaining further revealed that p65 nuclear translocation activated by RANKL was downregulated by sialidase, resulting in the sequestration of p65 in the cytoplasm (FIG. 28f, g). Western blot analysis of the subcellular distribution of p65 showed that RANKL-induced p65 nuclear translocation and cytosolic IκBα phosphorylation and that sialidase treatment dampened these effects (FIG. 28h). Cell-cell fusion is a fundamental cellular activity of macrophages, and multinucleation is a complex process that is likely involved in multiple signaling pathways. Activation of downstream signaling by both Siglec15 and sialylated TLR2 is involved in different cells simultaneously, and both signaling pathways could also occur in each cell, which could be the critical mechanism for cell fusion.

### Example VIII

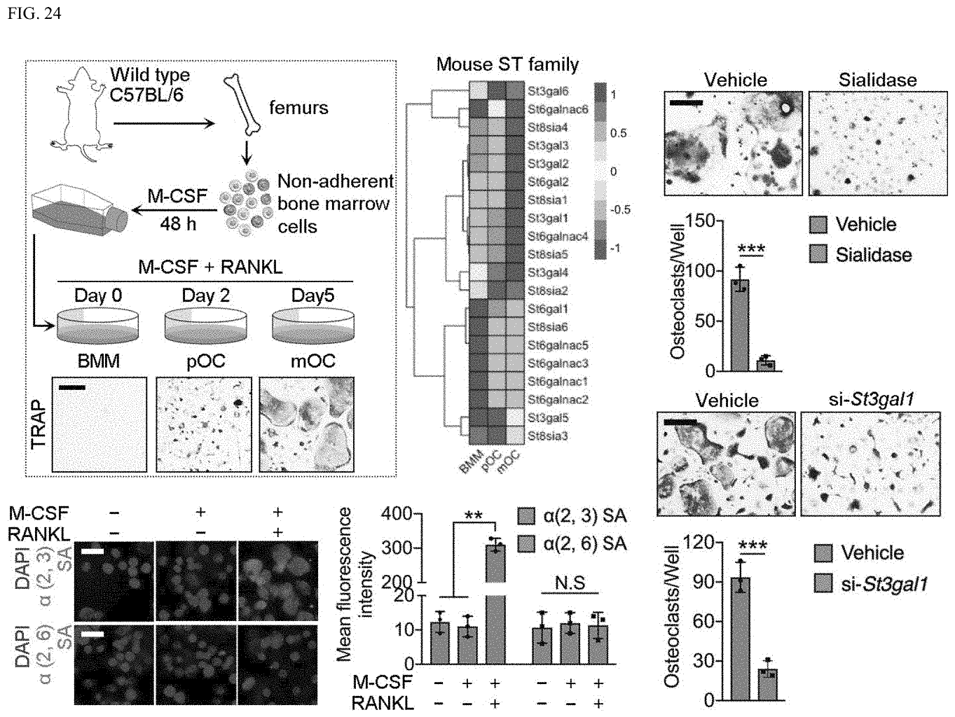

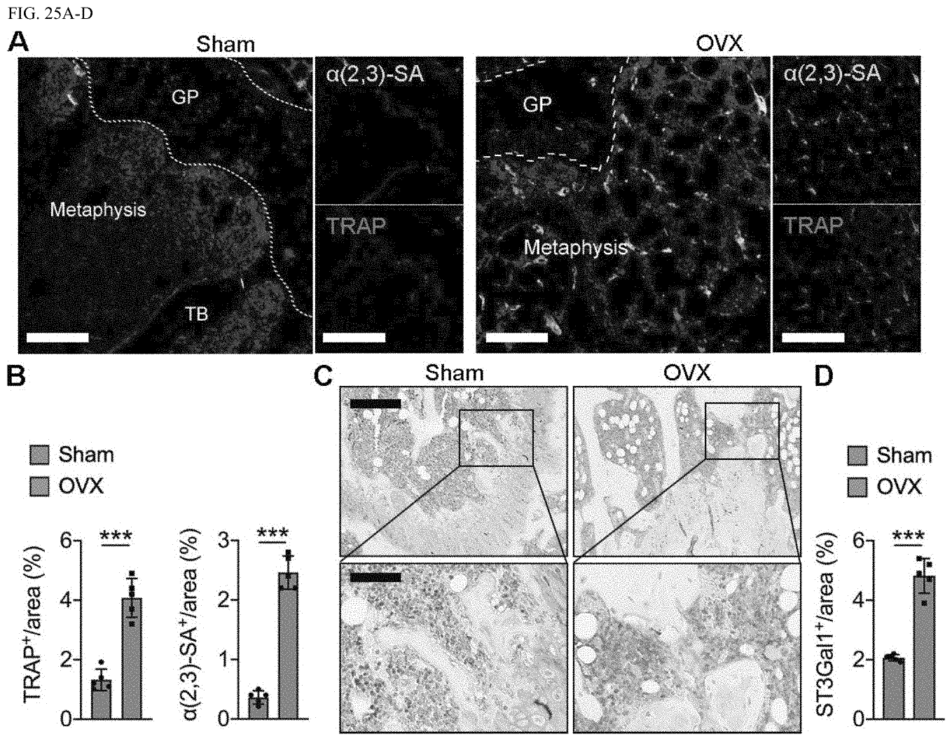

FIG. 24 shows that RANKL induces St3gal1 expression is required for osteoclast formation.

FIG. 25 shows that bone sialylation and St3Gal1 levels are increased in estrogen deficient osteoporosis.

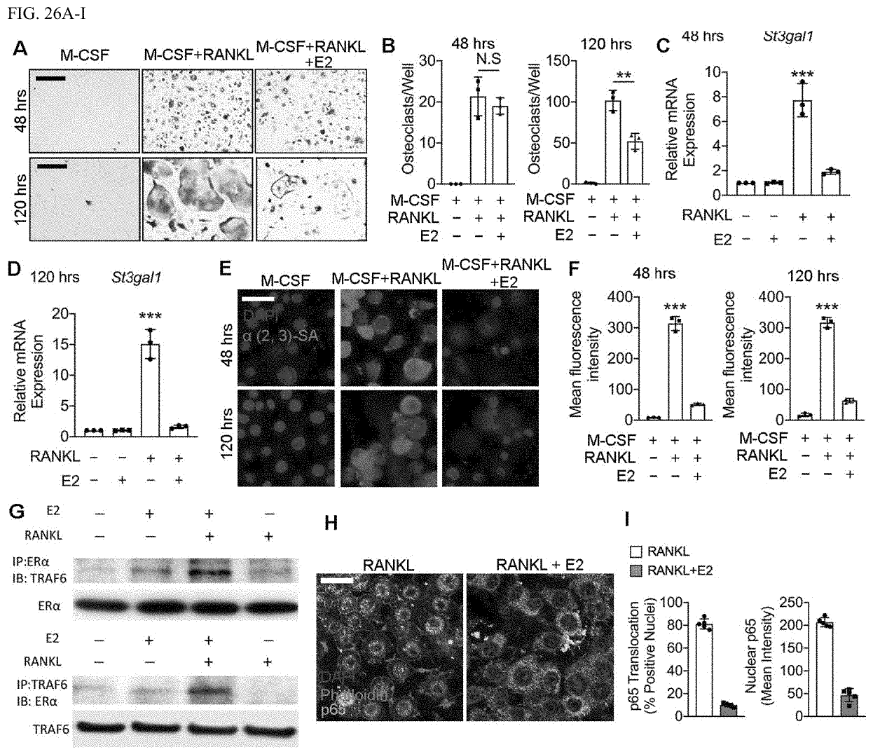

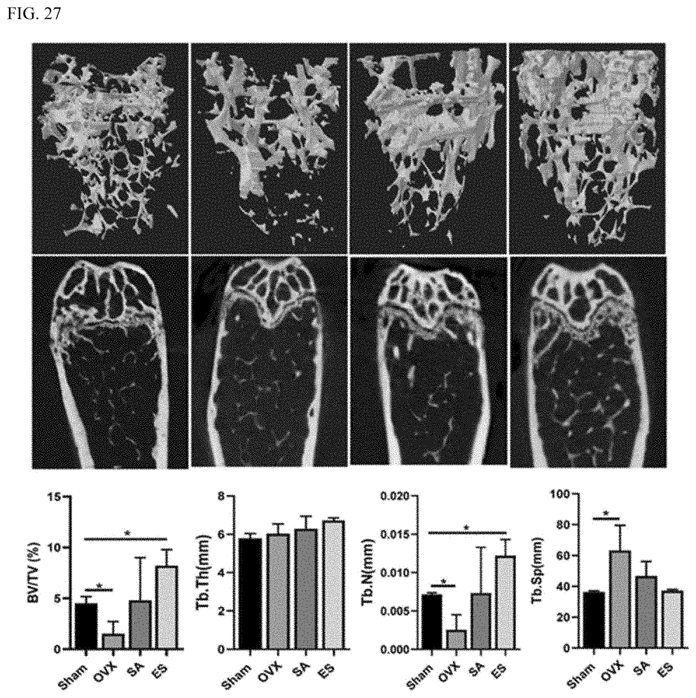

FIG. 26 shows that estrogen inhibits RANKL-activated St3gal1 transcription by repressing c-Fos Expression.

FIG. 27 shows that sialidase protects bone loss in OVX osteoporotic mice.

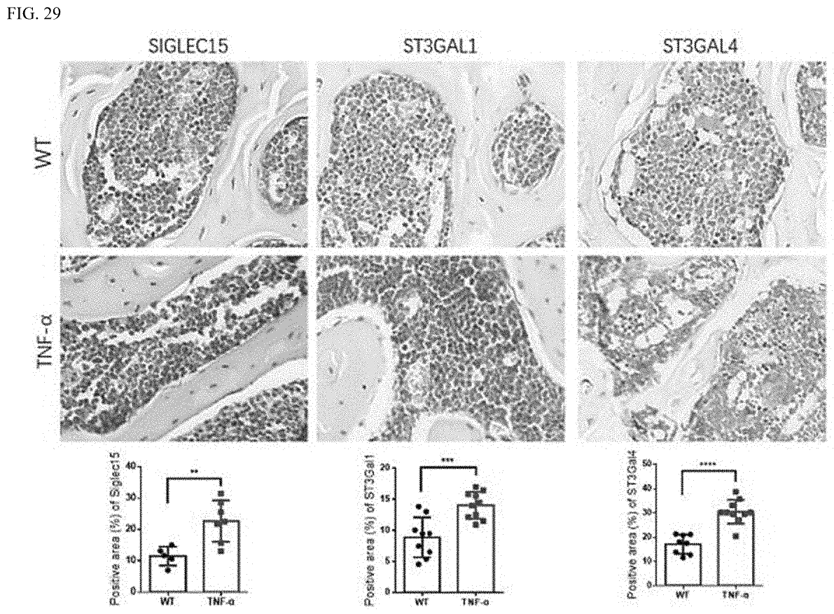

FIG. 29 shows that expression of both St3Gal1 and St3Gal4 are significantly elevated in human autoimmune diseases including rheumatoid arthritis and ankylosing spondylitis.

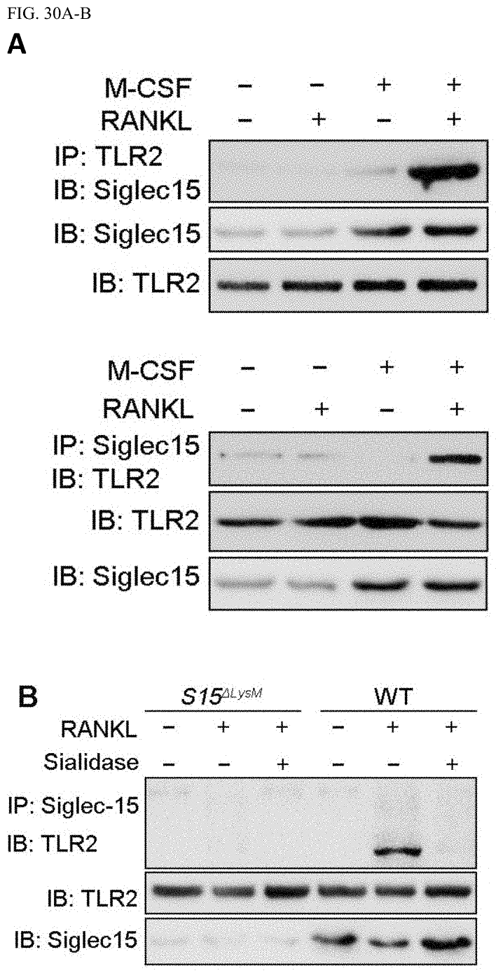

FIG. 30 shows sialylation is required for the binding between TLR2 and Siglec-15.

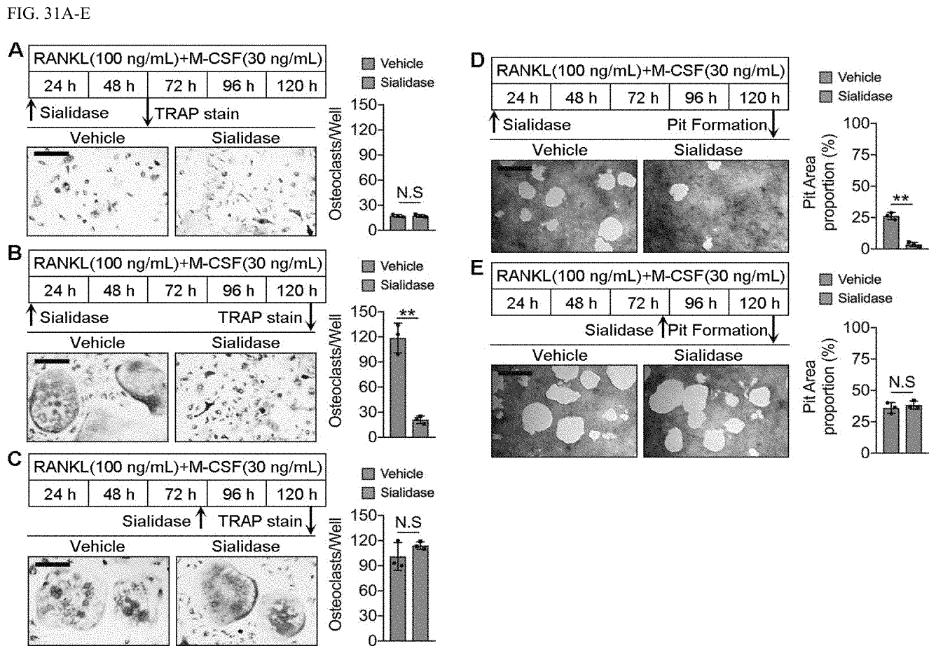

FIG. 31 shows sialidase blocks cell-cell fusion for osteoclast formation.

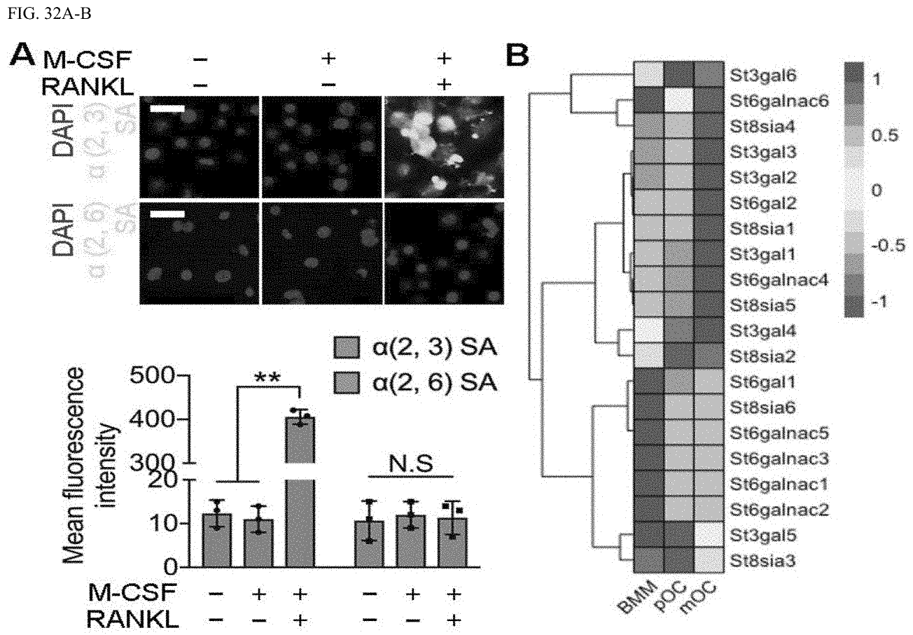

FIG. 32 shows sialyltransferase St3Gal1 is transcriptionally induced by RANKL.

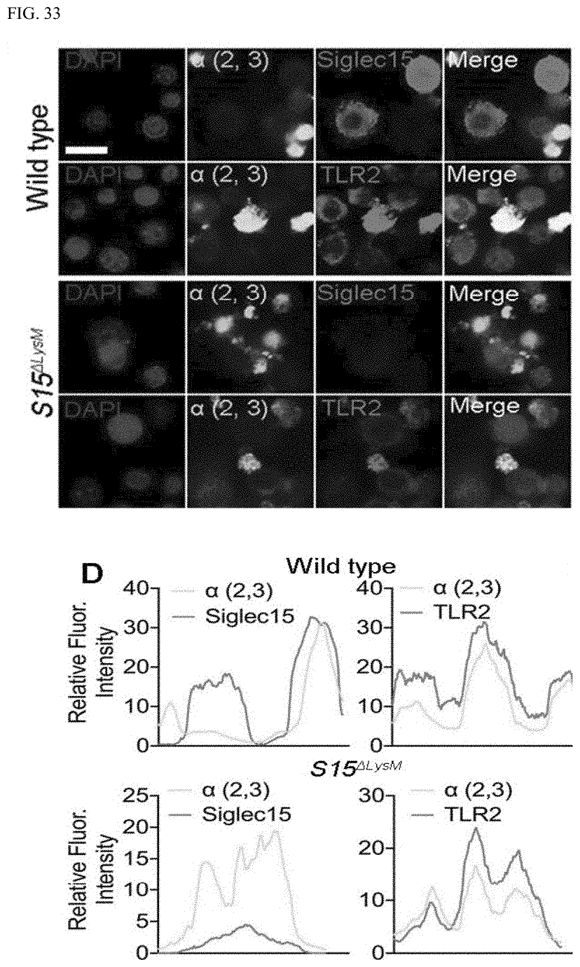

FIG. 33 shows α(2, 3) sialic acid is co-localized with TLR2.

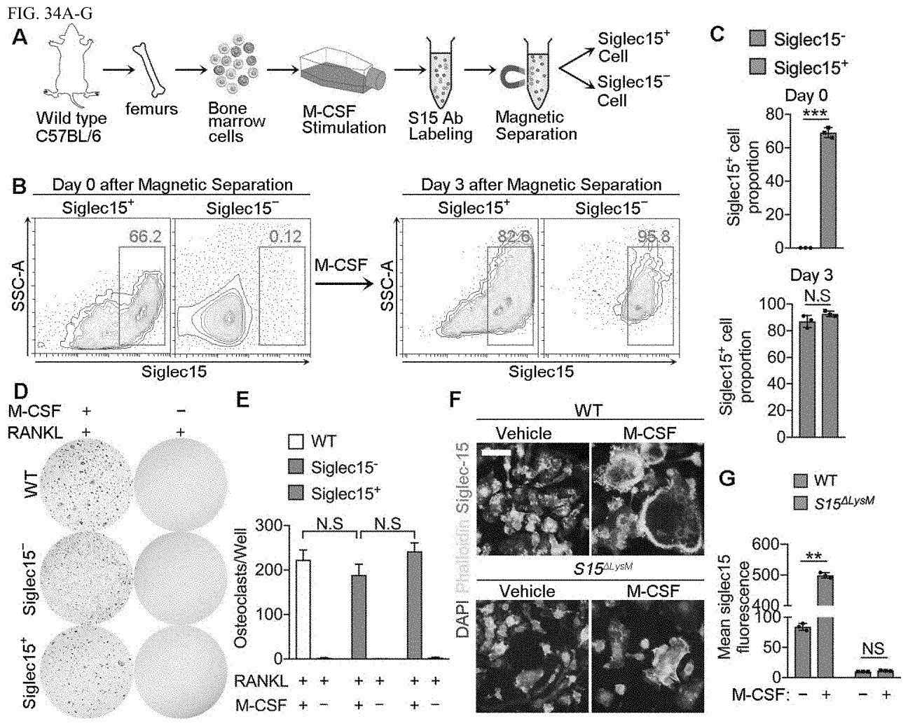

FIG. 34 shows Siglec15 expression is stimulated by M-CSF.

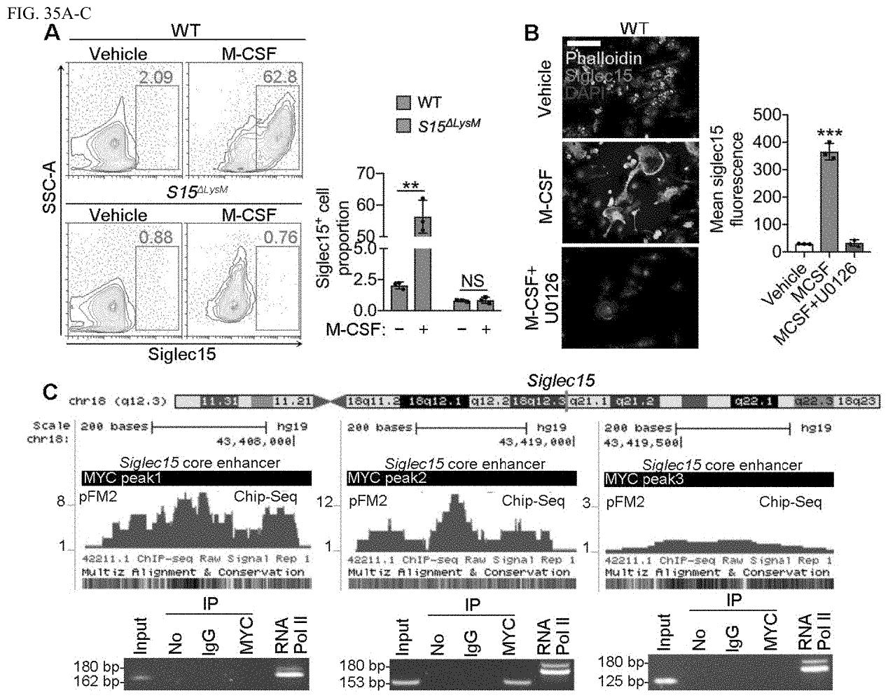

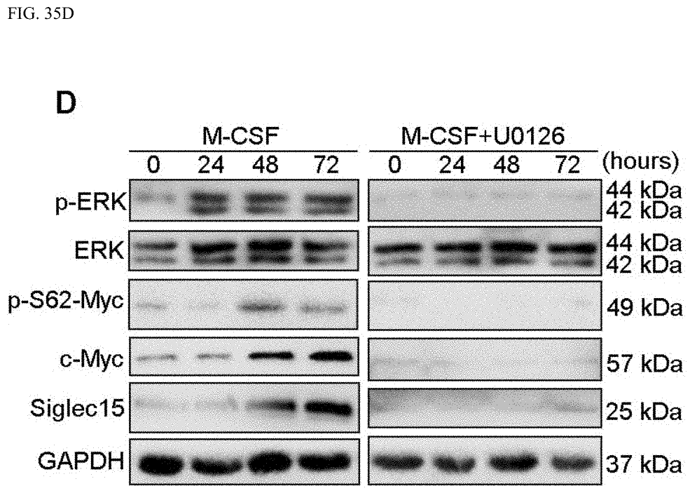

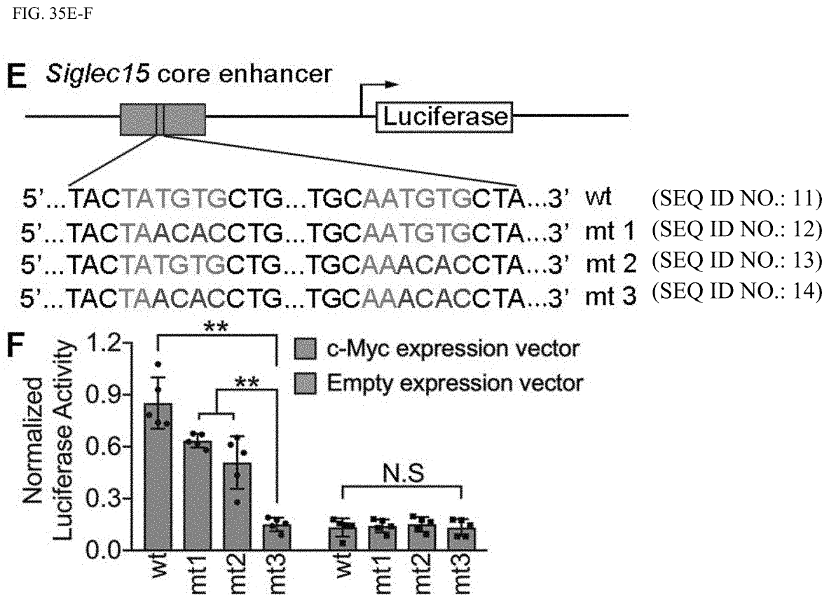

FIG. 35 shows M-CSF induced transcription of Siglec15 via MEK-ERK-MYC signaling.

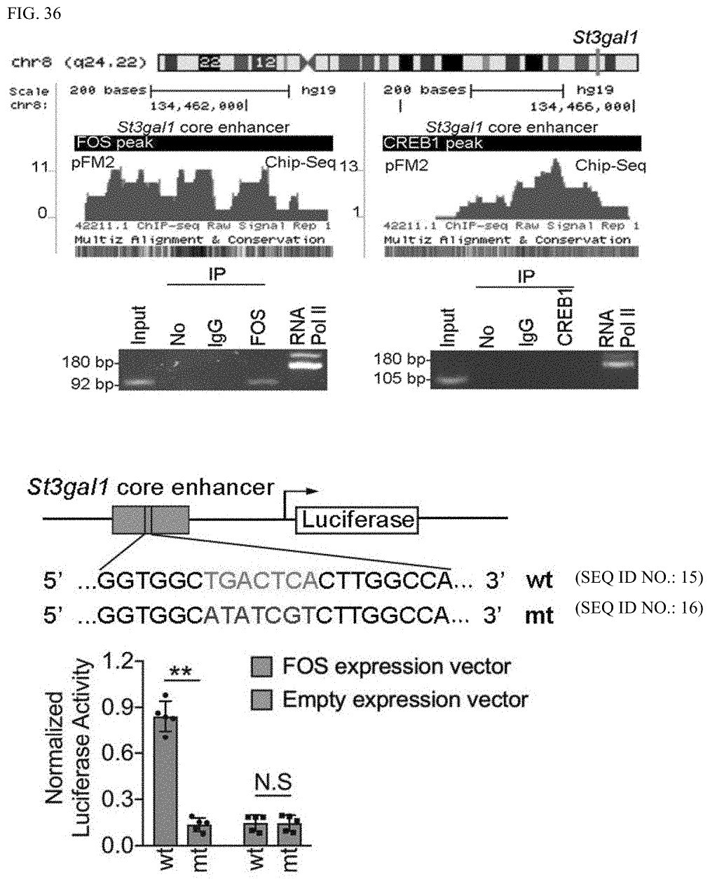

FIG. 36 shows RANKL Induces St3gal1 expression through Fos binding at its promoter.

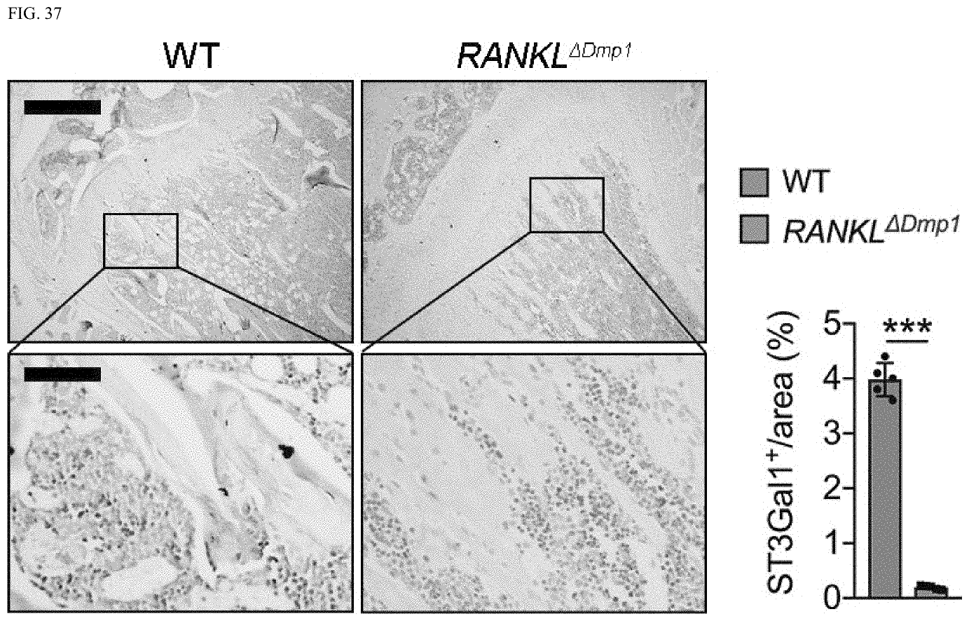

FIG. 37 shows St3 gal1 expression id abolished in RANKL^{ΔDmp} mice.

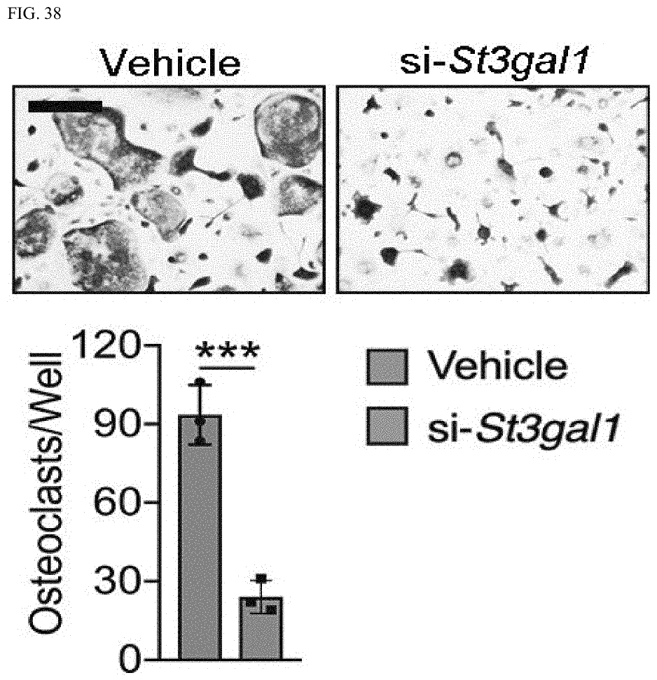

FIG. 38 shows siRNA St3gal1 blocked cell-cell fusion for osteoclast formation.

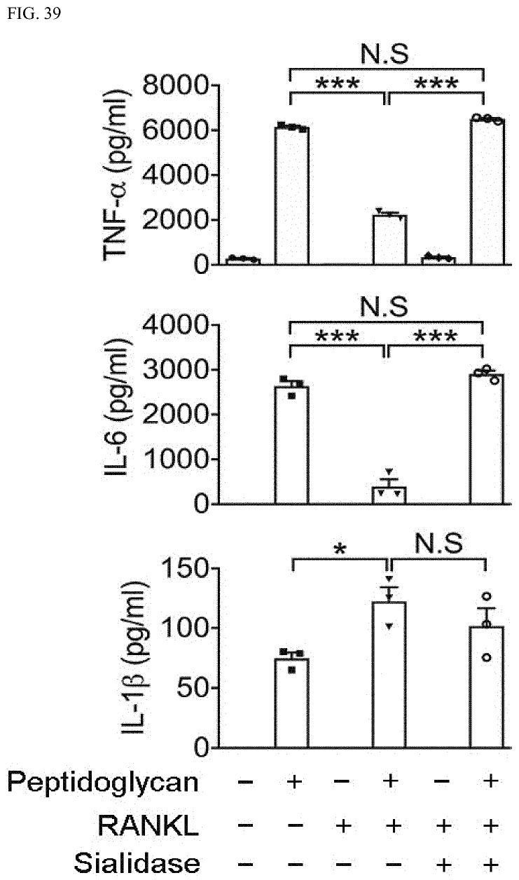

FIG. 39 shows sialidase inhibits RANKL effects on bone marrow macrophages.

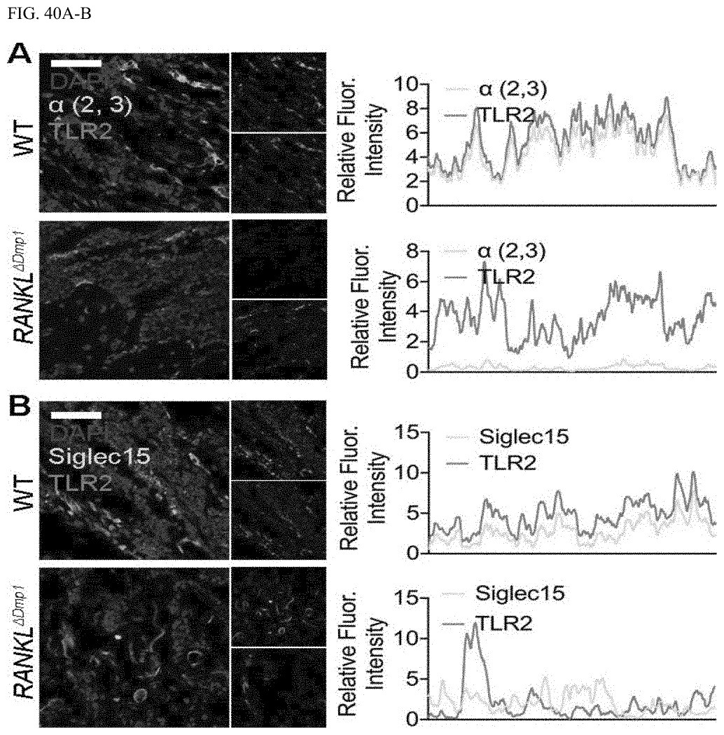

FIG. 40 shows knockout of RANKL in DMP-1 osteocytes disrupted α(2, 3) sialic acid co-localization with TLR2 and Siglec15.

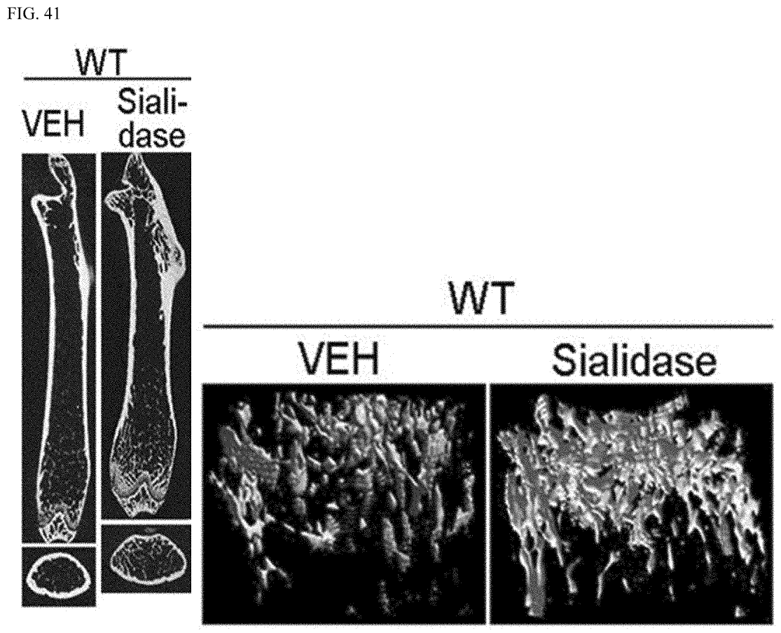

FIG. 41 shows removal of TLR2 α(2,3) sialic acid by sialidase increases bone mass in mice.

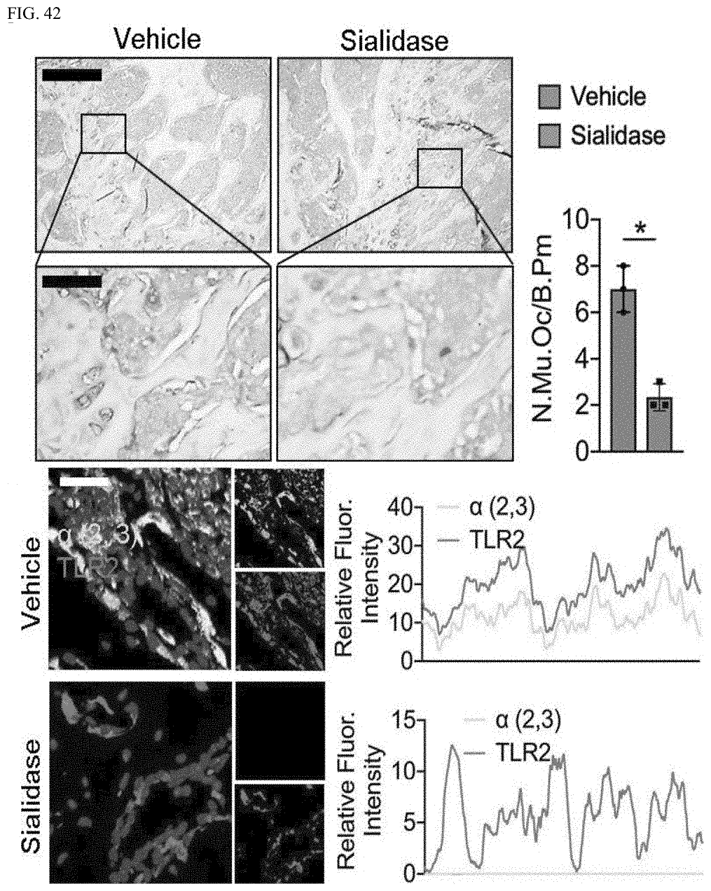

FIG. 42 shows removal of TLR2 α(2,3) sialic acid by sialidase increases bone mass in mice.

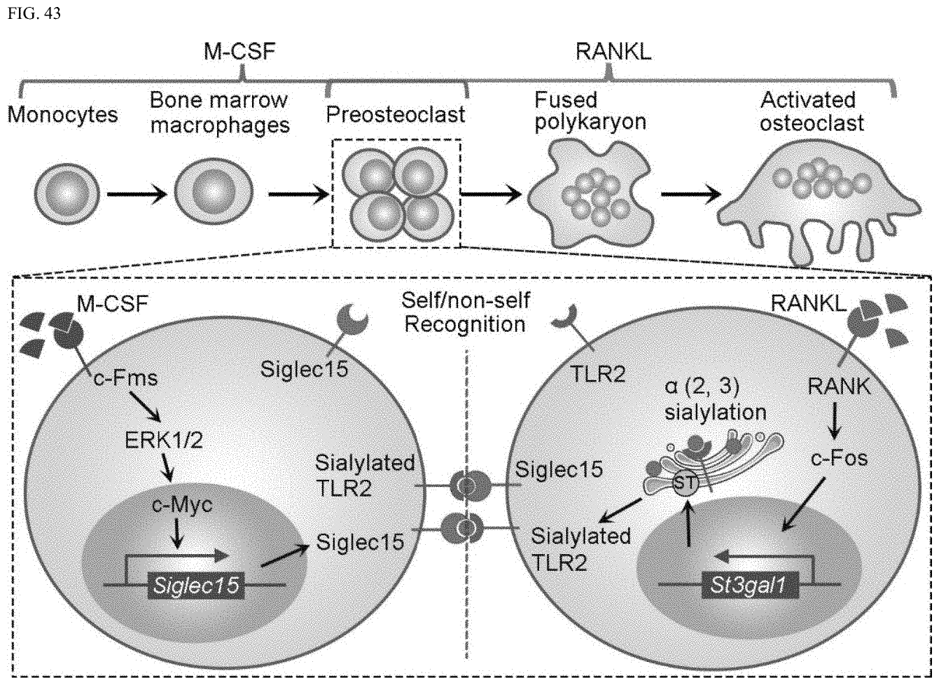

FIG. 43 shows a schematic diagram of Siglec15 binding to sialylated TLR2 for osteoclast fusion.

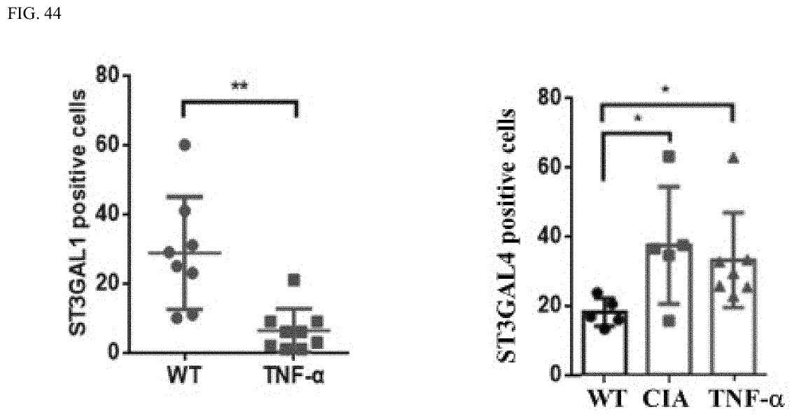

FIG. 44 shows sialyltransferase St3Gal4, not St3Gal1 is up-regulated in rheumatoid arthritis.

### Example VIII

This example provides the materials and methods utilized for Examples I-VII.

### Mice and Treatment

WT C57BL/6J, LysM-Cre, Dmp1-Cre, and RANKL^{ﬂ/ﬂ} mouse strains (referred to as “WT”) were purchased from the Jackson Laboratory (Ellsworth, ME, USA). Siglec-15^{ﬂ/ﬂ} mice were obtained from Dr. L. C. at Yale University. A Siglec-15 conditional knockout mouse strain was generated by crossing Siglec-15^{ﬂ/ﬂ} mice with LysM-Cre mice (referred to as Siglec15^{ΔLysM}) (Wang, 2019). A RANKL conditional knockout mouse strain was generated by crossing RANKL^{ﬂ/ﬂ} mice with Dmp1-Cre mice (referred to as RANKL^{ΔDmp1}) (Zhu, 2019). For time-course animal studies, WT littermates were used as controls, and male mice were euthanized with carbon dioxide asphyxiation for further analysis (10 to 12 per group). For sialidase injection, mice were anesthetized by intraperitoneal injection of ketamine (100 mg kg⁻¹) and xylazine (10 mg kg⁻¹). SialEXO 23 α2,3 specific sialidase (Genovis Inc, MA, USA) was prepared as 5 units preincubated in 20 mmol·L⁻¹ Tris pH 7.5 at 37° C. for 1 h and then injected intrafemorally. All the mice used in this study were maintained at the Johns Hopkins University School of Medicine animal facility. The animal study protocols were approved by the Animal Care and Use Committee of Johns Hopkins University.

RNA Sequencing

Bulk RNA-seq was used to screen the gene expression profiles of the mouse siglec family and TLR family. The detailed steps were described in a previous report (Ma, 2021). In brief, total RNA was isolated from BMMs and osteoclasts at different stages. After an initial quality check and purification, the transcripts were fragmented and converted into cDNA for library creation. An Illumina NovaSeq 6000 platform was used for sequencing.

### μCT Analysis

Male mice on different genetic backgrounds were used for analysis of bone phenotype. Carbon dioxide asphyxiation was used for mouse euthanasia. For μCT scanning, mouse femurs and tibias were dissected and fixed with paraformaldehyde for at least 24 h. A Bruker micro-CT Skyscan 1172 (Kontich, Belgium) system was used for scanning. The detailed scanning information, including isotropic voxel size, X-ray tube voltage, intensity, and exposure time, were described in our previous studies (Dou, 2021). In brief, 3D reconstruction of the region of interest in the mouse femur/tibia was realized by Nrecon (Kontich, Belgium). Contoured 2D images were analyzed using CT_{VOX} (Kontich, Belgium). Data analysis was performed using a CT analyzer (Kontich, Belgium).

### In Vitro Osteoclast Differentiation Assay

For TRAP staining, cells were first fixed using paraformaldehyde at 37° C. for 5 min before staining with a TRAP solution. The staining procedures were strictly performed according to the instructions of the manufacturer (Sigma-Aldrich, St. Louis, Mo., USA) before light microscopy observation. For sialidase treatment, 1 U mL⁻¹ SialEXO 23 α2,3 sialidase and 10 μmol·L⁻¹ U0126 were used for cell culture. The procedures were reported in detail in our previous studies (Dou, 2014; Dou, 2018). In brief, cells were washed, fixed, and permeabilized, followed by blocking. A primary antibody against vinculin (1:1 000) was incubated for 12h at 4° C. DAPI (1:2 000) was used for nuclear counterstaining. For the bone resorption assay, cells were incubated on bovine bone slices and then placed in 48-well plates for osteoclastic stimulation. The cells were removed from the slice surface with a bleach solution for further observation of pit formation.

### Immunohistochemistry, Immunofluorescence and Histomorphometry

Mouse bone specimens were first fixed and then decalcified using 10% EDTA (Sigma-Aldrich, St. Louis, Mo., USA) for 14 days with constant shaking. For the histological assays, the detailed protocols were reported in a previous study (Dou, J. Bone Min. Res. 33, 899-908 (2018)). In short, the samples were then dehydrated and embedded in optimal cutting temperature compound (Sakura Finetek, Torrance, Calif., USA) or in paraffin. Four-μm-thick coronal-oriented femur sections were prepared for TRAP staining. Forty-μm-thick coronal-oriented femur sections were prepared for IF staining. The detailed protocols were described in a previous study study (Dou, J. Bone Min. Res. 33, 899-908 (2018)). Briefly, the sections were incubated with primary antibodies against mouse TLR2 (Santa Cruz Biotechnology, sc-21759, 1:200), Siglec15 (PA5-48221, Thermo Fisher Scientific, 1:100), ST3GAL1 (PA5-21721, Thermo Fisher Scientific, 1:50), and TRAP (Abcam, ab191406, 1:100) for 12 h at 4° C. For sialic acid detection, biotinylated Maackia Amurensis Lectin II (MAL II) (Vector Laboratories, CA, USA) was used to label the α2,3 linkage, and biotinylated Sambucus Nigra Lectin (SNA) (Vector Laboratories, CA, USA) was used to label the α2,6 linkage. Fluorescein-conjugated streptavidin (Vector Laboratories, CA, USA) was used for the addition of a fluorescent label to biotinylated sialic acid conjugates. A Zeiss LSM 780 confocal microscope and an Olympus BX51 microscope were used for image capture.

### ChIP-PCR Assay

For the ChIP assay, primary BMMs were cultured with M-CSF or GM-CSF stimulation for 48 h to detect Siglec15 core enhancer DNA binding. M-CSF-primed cells were induced with RANKL for another 72 h to detect St3 gal1 core enhancer DNA binding. Afterward, the cells were cross-linked using 1% formaldehyde and lysed. DNA fragmentation was then achieved by enzymatic digestion with micrococcal nuclease (MNase). After digestion, 10% of the sample was preserved as the total input aliquot for further use. The remaining supernatant was then incubated with a ChIP-grade primary antibody against mouse p-CREB (Abcam, ab32096, 10 μg), c-FOS (Abcam, ab27793, 10 μg), or c-MYC (Abcam, ab9132, 10 μg). An anti-RNA Polymerase II antibody was used as the positive IP control, and normal rabbit IgG was used as the negative IP control. The supernatant was incubated overnight at 4° C. with mixing before immunoprecipitation using ChIP-grade Protein A/G Magnetic Beads following the suggestions of a ChIP kit (26157, Thermo Fisher Scientific). After elution, the DNA was then purified and recovered according to the manufacturer's instructions, and PCR detection was performed. The PCR primers used to detect MYC binding were as follows: Site #1, forward: 5′-TGCGGTGACTGATATACGCA-3′ (SEQ ID NO: 1), and reverse: 5′-ACCATTTTCTCTTGCTCGCG-3′ (SEQ ID NO: 2); Site #2, forward: 5′-GGTCACGGCTACCAGGTG-3′ (SEQ ID NO: 3), and reverse: 5′-GTGGAAGCGGAACAGGTAGA-3′ (SEQ ID NO: 4); and Site #3, forward: 5′-TGCGGTGACTGATATACGCA-3′ (SEQ ID NO: 5), and reverse: 5′-ACCATTTTCTCTTGCTCGCG-3′ (SEQ ID NO: 6). The PCR primers used to detect FOS binding were as follows: forward: 5′-GCCCAGTGACGTAGGAAGTC-3′ (SEQ ID NO: 7), and reverse: 5′-GTCGCGGTTGGAGTAGTAGG-3′ (SEQ ID NO: 8). The PCR primers used to detect CREB binding were as follows: forward: 5′-CAGCGAGCTGTGCCAGAC-3′ (SEQ ID NO: 9), and reverse: 5′-AACTCCACGCGGCAGAAGTA-3′ (SEQ ID NO: 10). ChIP positive control primers (GAPDH promoter) were provided in the kit (26157, Thermo Fisher Scientific). The PCR program comprised 40 cycles of 95° C. for 20 s, 62° C. for 60 s, and 72° C. for 30 s. For visualization and gel staining, 5 μL of PCR products was added to 1.5% agarose gels (Millipore Sigma, MO).

### Bioinformatics

The prediction of gene core enhancer transcription factor binding was achieved with the Encyclopedia of DNA Elements project, and ChIP-seq data were supported by the UCSC genome browser. The determination of Siglec15 ligand candidates was performed using a LC-MS dataset (ProteomeXchange Consortium, PXD006359), and the results were visualized using the R program. All raw data and processed bulk RNA-seq data were acquired from the GEO database (GSE133515).

### Co-Immunoprecipitation, Immunoblotting Analysis and Luciferase Reporter Assay

For the co-IP assays, cells were first lysed in the presence of protease inhibitors using IP buffer. The lysates were then immunoprecipitated using primary antibodies against mouse TLR2 (Santa Cruz Biotechnology, sc-21759) and Siglec15 (PA5-48221, Thermo Fisher Scientific), followed by absorption on Protein A/G as suggested by the manufacturer (26149, Thermo Fisher Scientific). SDS-PAGE was then used to separate the immunoprecipitates before transfer to a nitrocellulose membrane for immunoblotting procedures. The membranes were incubated with primary antibodies against mouse p-ERK (44-680G, Thermo Fisher Scientific, 1:1 000), ERK (13-6200, Thermo Fisher Scientific, 1:1 000), p-S62-Myc (ab51156, Abcam, 1:1 000), c-Myc (ab32072, Abcam, 1:1 000), TLR2 (Santa Cruz Biotechnology, sc-21759, 1:1 000), and Siglec15 (PA5-48221, Thermo Fisher Scientific, 1:1 000) for 12 h at 4° C., followed by a 1-h incubation with a secondary antibody (1:1 000). The Dual-Luciferase Reporter Assay System (Promega, Madison, Wis., USA) was used to detect luciferase activity. Enhancer assays were performed with adjustment for transfection efficiency differences (Cao, 2010). Reporter cotransfection assays were performed with a reporter-to-activator plasmid.

### Magnetic Microbead Cell Sorting and Flow Cytometry Analysis

To sort Siglec15⁺ cells, whole bone marrow was collected from mouse femurs and tibias after mice were euthanized with an overdose of inhaled isoflurane. Whole bone marrow cells were also used for culture and induction of BMMs using M-CSF (50 ng mL⁻¹) for higher Siglec15⁺ cell enrichment in further studies. After red blood cell lysis, the cell number was counted, and the same numbers of cells were incubated with a biotinylated mouse Siglec15-specific antibody (PA5-48221, Thermo Fisher Scientific). The primary antibody was biotinylated using a One-Step Antibody Biotinylation Kit (130-093-385, Miltenyi Biotec, MA) in accordance with the manufacturer's instructions. For Siglec15⁺ cell separation, cells were then labeled with a 1:5 dilution of MACS® anti-biotin microBeads (130-090-485, Miltenyi Biotec, MA) for 15 m. Each sample (8×10⁶ antibody- and microbead-labeled cells) was magnetically sorted at room temperature using “MS” columns inserted into a Miltenyi OctoMACS separator (130-042-201 and 130-042-109, Miltenyi Biotec). The cells were then placed on ice before counting. The Siglec15⁺ and Siglec15- cell distributions were analyzed and quantified by flow cytometry. Cells were incubated with a conjugated anti-mouse Siglec15 antibody (PA5-48221, Thermo Fisher Scientific) for 30 min on ice. The conjugation of the primary antibody was performed using an Atto633 Conjugation Kit (ab269898, Abcam) in accordance with the manufacturer's instructions. The cells were then sorted for Atto633 enrichment after live/dead cell sorting.

### Statistical Analysis

All data presented in this study were generated from at least three repeated assays unless otherwise indicated. SPSS software (Ver. 20.0) and Prism 8.0 software (GraphPad) were used for statistical analysis. Differences were considered statistically significant at P<0.05. Error bars in the plots represent the standard deviation (SD).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes. In particular, the following references are denoted within the specification and are herein incorporated by reference in their entireties:
Alturfan, A. A., Uslu, E., Alturfan, E. E., Hatemi, G., Fresko, I., and Kokoglu, E. (2007). Increased serum sialic acid levels in primary osteoarthritis and inactive rheumatoid arthritis. Tohoku J Exp Med 213, 241-248.
Amith, S. R., Jayanth, P., Franchuk, S., Finlay, T., Seyrantepe, V., Beyaert, R., Pshezhetsky, A. V., and Szewczuk, M. R. (2010). Neul desialylation of sialyl alpha-2,3-linked beta-galactosyl residues of TOLL-like receptor 4 is essential for receptor activation and cellular signaling. Cell Signal 22, 314-324.
Baroukh, B., Cherruau, M., Dobigny, C., Guez, D., and Saffar, J. L. (2000). Osteoclasts differentiate from resident precursors in an in vivo model of synchronized resorption: a temporal and spatial study in rats. Bone 27, 627-634.
Boyle, W. J., Simonet, W. S., and Lacey, D. L. (2003). Osteoclast differentiation and activation. Nature 423, 337-342.
Briard, J. G., Jiang, H., Moremen, K. W., Macauley, M. S., and Wu, P. (2018). Cell-based glycan arrays for probing glycan-glycan binding protein interactions. Nat Commun 9, 880.
Bull, C. et al. Sialic acid blockade suppresses tumor growth by enhancing T-cell-mediated tumor immunity. Cancer Res. 78, 3574-3588 (2018).
Cao, L. et al. Genome-wide identification of PAX3-FKHR binding sites in rhabdomyosarcoma reveals candidate target genes important for development and cancer. Cancer Res. 70, 6497-6508 (2010).
Carmona-Fernandes, D., Santos, M. J., Perpetuo, I. P., Fonseca, J. E., and Canhao, H. (2011). Soluble receptor activator of nuclear factor kappaB ligand/osteoprotegerin ratio is increased in systemic lupus erythematosus patients. Arthritis Res Ther 13, R175.
Chang, L., Chen, Y. J., Fan, C. Y., Tang, C. J., Chen, Y. H., Low, P. Y., Ventura, A., Lin, C. C., Chen, Y. J., and Angata, T. (2017). Identification of Siglec Ligands Using a Proximity Labeling Method. J Proteome Res 16, 3929-3941.
Chatani, M., Takano, Y., and Kudo, A. (2011). Osteoclasts in bone modeling, as revealed by in vivo imaging, are essential for organogenesis in fish. Dev Biol 360, 96-109.
Chen, G. Y., Brown, N. K., Wu, W., Khedri, Z., Yu, H., Chen, X., van de Vlekkert, D., D'Azzo, A., Zheng, P., and Liu, Y. (2014). Broad and direct interaction between TLR and Siglec families of pattern recognition receptors and its regulation by Neul. Elife 3, e04066.
Crean, S. M., Meneski, J. P., Hullinger, T. G., Reilly, M. J., DeBoever, E. H., and Taichman, R. S. (2004). N-linked sialyated sugar receptors support haematopoietic cell-osteoblast adhesions. Br J Haematol 124, 534-546.
Dai, X. M., Ryan, G. R., Hapel, A. J., Dominguez, M. G., Russell, R. G., Kapp, S., Sylvestre, V., and Stanley, E. R. (2002). Targeted disruption of the mouse colony-stimulating factor 1 receptor gene results in osteopetrosis, mononuclear phagocyte deficiency, increased primitive progenitor cell frequencies, and reproductive defects. Blood 99, 111-120.
Dou, C. et al. MiR-7b directly targets DC-STAMP causing suppression of NFATc1 and c-Fos signaling during osteoclast fusion and differentiation. Biochim. Biophys. Acta 1839, 1084-1096 (2014).
Dou, C. et al. Graphene-based MicroRNA transfection blocks preosteoclast fusion to increase bone formation and vascularization. Adv. Sci. 5, 1700578 (2018).
Dou, C. et al. Estrogen deficiency-mediated M2 macrophage osteoclastogenesis contributes to M1/M2 ratio alteration in ovariectomized osteoporotic mice. J Bone Min. Res. 33, 899-908 (2018).
Guccione, E., Martinato, F., Finocchiaro, G., Luzi, L., Tizzoni, L., Dall' Olio, V., Zardo, G., Nervi, C., Bernard, L., and Amati, B. (2006). Myc-binding-site recognition in the human genome is determined by chromatin context. Nat Cell Biol 8, 764-770.
Hernandez, R. K., Wade, S. W., Reich, A., Pirolli, M., Liede, A., and Lyman, G. H. (2018). Incidence of bone metastases in patients with solid tumors: analysis of oncology electronic medical records in the United States. BMC Cancer 18, 44.
Hiruma, Y., Hirai, T., and Tsuda, E. (2011). Siglec-15, a member of the sialic acid-binding lectin, is a novel regulator for osteoclast differentiation. Biochem Biophys Res Commun 409, 424-429.
Hiruma, Y., Tsuda, E., Maeda, N., Okada, A., Kabasawa, N., Miyamoto, M., Hattori, H., and Fukuda, C. (2013). Impaired osteoclast differentiation and function and mild osteopetrosis development in Siglec-15-deficient mice. Bone 53, 87-93.
Jin, B., Sun, T., Yu, X. H., Yang, Y. X. & Yeo, A. E. The effects of TLR activation on T-cell development and differentiation. Clin. Dev. Immunol. 2012, 836485 (2012).
Kameda, Y., Takahata, M., Komatsu, M., Mikuni, S., Hatakeyama, S., Shimizu, T., Angata, T., Kinjo, M., Minami, A., and Iwasaki, N. (2013). Siglec-15 regulates osteoclast differentiation by modulating RANKL-induced phosphatidylinositol 3-kinase/Akt and Erk pathways in association with signaling Adaptor DAP12. J Bone Miner Res 28, 2463-2475.
Kawasaki, N., Rademacher, C., and Paulson, J. C. (2011). CD22 regulates adaptive and innate immune responses of B cells. J Innate Immun 3, 411-419.
Keppler, O. T., Peter, M. E., Hinderlich, S., Moldenhauer, G., Stehling, P., Schmitz, I., Schwartz-Albiez, R., Reutter, W., and Pawlita, M. (1999). Differential sialylation of cell surface glycoconjugates in a human B lymphoma cell line regulates susceptibility for CD95 (APO-1/Fas)-mediated apoptosis and for infection by a lymphotropic virus. Glycobiology 9, 557-569.
Koga, T., Inui, M., Inoue, K., Kim, S., Suematsu, A., Kobayashi, E., Iwata, T., Ohnishi, H., Matozaki, T., Kodama, T., et al. (2004). Costimulatory signals mediated by the ITAM motif cooperate with RANKL for bone homeostasis. Nature 428, 758-763.
Komai-Koma, M., Jones, L., Ogg, G. S., Xu, D. & Liew, F. Y. TLR2 is expressed on activated T cells as a costimulatory receptor. Proc. Natl Acad. Sci. U.S.A. 101, 3029-3034 (2004).
Krisher, T., and Bar-Shavit, Z. (2014). Regulation of osteoclastogenesis by integrated signals from toll-like receptors. J Cell Biochem 115, 2146-2154. Lacey, D. L., Timms, E., Tan, H. L., Kelley, M. J., Dunstan, C. R., Burgess, T., Elliott, R., Colombero, A., Elliott, G., Scully, S., et al. (1998). Osteoprotegerin ligand is a cytokine that regulates osteoclast differentiation and activation. Cell 93, 165-176.
Lacey, D. L. et al. Osteoprotegerin ligand is a cytokine that regulates osteoclast differentiation and activation. Cell 93, 165-176 (1998).
Levaot, N., Ottolenghi, A., Mann, M., Guterman-Ram, G., Kam, Z., and Geiger, B. (2015). Osteoclast fusion is initiated by a small subset of RANKL-stimulated monocyte progenitors, which can fuse to RANKL-unstimulated progenitors. Bone 79, 21-28.
Li, W., Liu, Y., Zheng, X., Gao, J., Wang, L., and Li, Y. (2019). Investigation of the Potential Use of Sialic Acid as a Biomarker for Rheumatoid Arthritis. Ann Clin Lab Sci 49, 224-231.
Liou, L. B., and Huang, C. C. (2016). Sialyltransferase and Neuraminidase Levels/Ratios and Sialic Acid Levels in Peripheral Blood B Cells Correlate with Measures of Disease Activity in Patients with Systemic Lupus Erythematosus and Rheumatoid Arthritis: A Pilot Study. PLoS One 11, e0151669.
Ma, Q. et al. Osteoclast-derived apoptotic bodies couple bone resorption and formation in bone remodeling. Bone Res. 9, 5 (2021).
Manolagas, S. C. (2000). Birth and death of bone cells: basic regulatory mechanisms and implications for the pathogenesis and treatment of osteoporosis. Endocr Rev 21, 115-137.
McDonald, M. M. et al. Osteoclasts recycle via osteomorphs during RANKL-stimulated bone resorption. Cell 184, 1330-1347 e1313 (2021).
Medzhitov, R. Toll-like receptors and innate immunity. Nat. Rev. Immunol. 1, 135-145 (2001).
Ochareon, P., and Herring, S. W. (2011). Cell replication in craniofacial periosteum: appositional vs. resorptive sites. J Anat 218, 285-297.
Pacifici, R. (1996). Estrogen, cytokines, and pathogenesis of postmenopausal osteoporosis. J Bone Miner Res 11, 1043-1051.
Pettit, A. R., Ji, H., von Stechow, D., Muller, R., Goldring, S. R., Choi, Y., Benoist, C., and Gravallese, E. M. (2001). TRANCE/RANKL knockout mice are protected from bone erosion in a serum transfer model of arthritis. Am J Pathol 159, 1689-1699.
Ross, F. P., and Teitelbaum, S. L. (2005). alphavbeta3 and macrophage colony-stimulating factor: partners in osteoclast biology. Immunol Rev 208, 88-105.
Sato, K., Suematsu, A., Okamoto, K., Yamaguchi, A., Morishita, Y., Kadono, Y., Tanaka, S., Kodama, T., Akira, S., Iwakura, Y., et al. (2006). Thl7 functions as an osteoclastogenic helper T cell subset that links T cell activation and bone destruction. J Exp Med 203, 2673-2682.
Schultz, M. J., Swindall, A. F., and Bellis, S. L. (2012). Regulation of the metastatic cell phenotype by sialylated glycans. Cancer Metastasis Rev 31, 501-518.
Stamatos, N. M., Curreli, S., Zella, D., and Cross, A. S. (2004). Desialylation of glycoconjugates on the surface of monocytes activates the extracellular signal-related kinases ERK ½ and results in enhanced production of specific cytokines. J Leukoc Biol 75, 307-313.
Stuible, M., Moraitis, A., Fortin, A., Saragosa, S., Kalbakji, A., Filion, M., and Tremblay, G. B. (2014). Mechanism and function of monoclonal antibodies targeting siglec-15 for therapeutic inhibition of osteoclastic bone resorption. J Biol Chem 289, 6498-6512.
Takahata, M., Iwasaki, N., Nakagawa, H., Abe, Y., Watanabe, T., Ito, M., Majima, T., and Minami, A. (2007). Sialylation of cell surface glycoconjugates is essential for osteoclastogenesis. Bone 41, 77-86.
Takami, M., Kim, N., Rho, J., and Choi, Y. (2002). Stimulation by toll-like receptors inhibits osteoclast differentiation. J Immunol 169, 1516-1523.
Takamiya, R., Ohtsubo, K., Takamatsu, S., Taniguchi, N. & Angata, T. The interaction between Siglec-15 and tumor-associated sialyl-Tn antigen enhances TGF-beta secretion from monocytes/macrophages through the DAP12-Syk pathway. Glycobiology 23, 178-187 (2013).
Teoh, S. T., Ogrodzinski, M. P., Ross, C., Hunter, K. W., and Lunt, S. Y. (2018). Sialic Acid Metabolism: A Key Player in Breast Cancer Metastasis Revealed by Metabolomics. Front Oncol 8, 174.
Urban-Wojciuk, Z. et al. The role of TLRs in anti-cancer immunity and tumor rejection. Front. Immunol. 10, 2388 (2019).
Varki, A. Sialic acids in human health and disease. Trends Mol. Med. 14, 351-360 (2008).
Vijay, G. S., Lodha, S. K., Sareen, P. M. & Vijay, R. K. Serum sialic acid estimation in various bone disorders. J. Indian Med. Assoc. 79, 109-110 (1982).
Wang, J., Sun, J., Liu, L. N., Flies, D. B., Nie, X., Toki, M., Zhang, J., Song, C., Zarr, M., Zhou, X., et al. (2019). Siglec-15 as an immune suppressor and potential target for normalization cancer immunotherapy. Nat Med 25, 656-666.
Weber, A. N., Morse, M. A., and Gay, N.J. (2004). Four N-linked glycosylation sites in human toll-like receptor 2 cooperate to direct efficient biosynthesis and secretion. J Biol Chem 279, 34589-34594.
Witten, P. E., and Huysseune, A. (2009). A comparative view on mechanisms and functions of skeletal remodelling in teleost fish, with special emphasis on osteoclasts and their function. Biol Rev Camb Philos Soc 84, 315-346.
Wu, Y., Ren, D., and Chen, G. Y. (2016). Siglec-E Negatively Regulates the Activation of TLR4 by Controlling Its Endocytosis. J Immunol 197, 3336-3347.
Xie, H., Cui, Z., Wang, L., Xia, Z., Hu, Y., Xian, L., Li, C., Xie, L., Crane, J., Wan, M., et al. (2014). PDGF-BB secreted by preosteoclasts induces angiogenesis during coupling with osteogenesis. Nat Med 20, 1270-1278.
Yasuda, H., Shima, N., Nakagawa, N., Yamaguchi, K., Kinosaki, M., Mochizuki, S., Tomoyasu, A., Yano, K., Goto, M., Murakami, A., et al. (1998). Osteoclast differentiation factor is a ligand for osteoprotegerin/osteoclastogenesis-inhibitory factor and is identical to TRANCE/RANKL. Proc Natl Acad Sci USA 95, 3597-3602.
Yoshida, H., Hayashi, S., Kunisada, T., Ogawa, M., Nishikawa, S., Okamura, H., Sudo, T., Shultz, L. D., and Nishikawa, S. (1990). The murine mutation osteopetrosis is in the coding region of the macrophage colony stimulating factor gene. Nature 345, 442-444.
Zanin-Zhorov, A. & Cohen, I. R. Signaling via TLR2 and TLR4 directly down-regulates T cell effector functions: the regulatory face of danger signals. Front. Immunol. 4, 211 (2013).
Zhang, C., Yan, L., Song, H., Ma, Z., Chen, D., Yang, F., Fang, L., Li, Z., Li, K., Li, D., et al. (2019). Elevated Serum Sialic Acid Levels Predict Prostate Cancer As Well As Bone Metastases. J Cancer 10, 449-457.
Zhu, S. et al. Subchondral bone osteoclasts induce sensory innervation and osteoarthritis pain. J Clin. Investig. 129, 1076-1093 (2019).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating, delaying progression of, or reducing the severity of a disorder characterized with increased osteoclast activity and/or increased Siglec15 and sialylated TLR (e.g., TLR2, TLR4) interaction, comprising administering to a subject in need thereof a therapeutically effective amount of an agent capable of hindering and/or inhibiting osteoclastogenesis and/or osteoclast activity through hindering and/or inhibiting Siglec15 and sialylated TLR (e.g., TLR2, TLR4) interaction.

2. The method of claim 1, wherein the administration results in one or more of the following: inhibition of Siglec15 activity and/or expression; inhibition of osteoclast precursor activation of Siglec15 expression; inhibition of M-CSF activity and/or expression thereby preventing osteoclast precursor activation of Siglec15 expression; inhibition of sialyation of TLR (e.g., TLR2, TLR4) thereby inhibiting interaction between Siglec15 and TLR (e.g., TLR2, TLR4); inhibition of RANKL stimulation of a sialytransferase (e.g., ST3Gal1, ST3Gal4) thereby inhibiting transfer of sialic acid to TLR (e.g., TLR2, TLR4) from such a sialytransferase (e.g., ST3Gal1, ST3Gal4); and inhibition of transfer of sialic acid to TLR (e.g., TLR2, TLR4) from a sialytransferase (e.g., ST3Gal1, ST3Gal4).

3. The method of claim 1, wherein the disorder characterized with increased osteoclast activity and/or increased Siglec15 and sialylated TLR (e.g., TLR2, TLR4) interaction is one or more of the following disorders: osteoporosis, rheumatoid arthritis, bone destruction accompanying rheumatoid arthritis, hypercalcemia, hypocalcemia, cancerous hypercalcemia, bone destruction accompanying multiple myeloma or cancer metastasis to bone, giant cell tumor, tooth loss due to periodontitis, osteolysis around a prosthetic joint, osteomyelitis, Paget's disease, ankylosing spondylitis, renal osteodystrophy, osteogenesis imperfecta, childhood osteoporosis, osteomalacia, bone necrosis, metastatic bone diseases, myeloma, fibrous dysplasia, aplastic bone diseases, metabolic bone diseases, and bone loss with age.

4. The method of claim 1, wherein the agent configured to inhibit and/or diminish Siglec15 and sialylated TLR (e.g., TLR2, TLR4) interaction is a sialytransferase inhibiting agent.

5. The method of claim 4, wherein the sialytransferase inhibiting agent is capable of inhibiting one or both of ST3Gal1 and ST3Gal4.

6. The method of claim 1, wherein the subject is a mammalian subject.

7. The method of claim 1, wherein the subject is a human patient suffering from or at risk of suffering from a disorder characterized with increased osteoclast activity and/or increased Siglec15 and sialylated TLR (e.g., TLR2, TLR4) interaction.

8. The method of claim 1, wherein the agent is co-administered with a drug known for treating a disorder characterized with increased osteoclast activity and/or increased Siglec15 and sialylated TLR (e.g., TLR2, TLR4) interaction.

9. A method of inhibiting and/or reducing Siglec15 activity and/or expression within cells comprising exposing cells characterized with increased Siglec15 activity and/or expression (compared to an established normal activity and/or expression level) a therapeutically effective amount of an agent configured to inhibit and/or diminish Siglec15 activity and/or expression within cells.

10. The method of claim 9, wherein the agent capable of inhibiting Siglec15 activity and/or expression is a sialytransferase inhibitor.

11. The method of claim 10, wherein the agent is capable of inhibiting ST3Gal1 expression and/or activity, ST3Gal4 expression and/or activity, or both ST3Gal1 and ST3Gal4 expression and/or activity.

12. A method of inhibiting and/or reducing sialyation of TLR (e.g., TLR2, TLR4) within cells comprising exposing cells characterized with increased sialyation of TLR (e.g., TLR2, TLR4) (compared to an established norm) a therapeutically effective amount of an agent configured to inhibit and/or diminish sialyation of TLR (e.g., TLR2, TLR4) within cells.

13. The method of claim 12, wherein the agent capable of inhibiting sialyation of TLR (e.g., TLR2, TLR4) is a sialytransferase inhibitor.

14. The method of claim 12, wherein the agent is capable of inhibiting ST3Gal1 expression and/or activity, ST3Gal4 expression and/or activity, or both ST3Gal1 and ST3Gal4 expression and/or activity.


SEQUENCE LISTING (16 sequences)

SEQ ID NO: 1 - protein, 20 aa, synthetic construct
TGCGGTGACTGATATACGCA

SEQ ID NO: 2 - protein, 20 aa, synthetic construct
ACCATTTTCTCTTGCTCGCG

SEQ ID NO: 3 - protein, 18 aa, synthetic construct
GGTCACGGCTACCAGGTG

SEQ ID NO: 4 - protein, 20 aa, synthetic construct
GTGGAAGCGGAACAGGTAGA

SEQ ID NO: 5 - protein, 20 aa, synthetic construct
TGCGGTGACTGATATACGCA

SEQ ID NO: 6 - protein, 20 aa, synthetic construct
ACCATTTTCTCTTGCTCGCG

SEQ ID NO: 7 - protein, 20 aa, synthetic construct
GCCCAGTGACGTAGGAAGTC

SEQ ID NO: 8 - protein, 20 aa, synthetic construct
GTCGCGGTTGGAGTAGTAGG

SEQ ID NO: 9 - protein, 18 aa, synthetic construct
CAGCGAGCTGTGCCAGAC

SEQ ID NO: 10 - protein, 20 aa, synthetic construct
AACTCCACGCGGCAGAAGTA

SEQ ID NO: 11 - protein, 24 aa, synthetic construct
TACTATGTGCTGTGCAATGTGCTA

SEQ ID NO: 12 - protein, 24 aa, synthetic construct
TACTAACACCTGTGCAATGTGCTA

SEQ ID NO: 13 - protein, 24 aa, synthetic construct
TACTATGTGCTGTGCAAACACCTA

SEQ ID NO: 14 - protein, 24 aa, synthetic construct
TACTAACACCTGTGCAAACACCTA

SEQ ID NO: 15 - protein, 21 aa, synthetic construct
GGTGGCTGACTCACTTGGCCA

SEQ ID NO: 16 - protein, 21 aa, synthetic construct
GGTGGCATATCGTCTTGGCCA